(19) United States
(12) Patent Application Publication
(10) Pub. No.: US 2016/0199316 A1
(43) Pub. Date: Jul. 14, 2016

(54) THREE-LAYER TRANSDERMAL THERAPY SYSTEM (TTS)

(71) Applicant: TESA LABTEC GMBH, Langenfeld (DE)

(72) Inventors: Armin BREITENBACH, Leverkusen (DE); Michael HORSTMANN, Neuwied (DE); Sebastian BRAUN, Wermelskirchen (DE); Ulrich BECKER, Steinen (DE)

(21) Appl. No.: 14/897,942

(22) PCT Filed: Jun. 16, 2014
(86) PCT No.: PCT/EP2014/001638

§ 371 Date: Mar. 8, 2016

(30) Foreign Application Priority Data
Jun. 14, 2013 (DE) 13172100.3

(51) Int. Cl. A61K 9/70; A61M 37/00; A61K 47/34; A61K 31/381; A61K 47/32

(52) U.S. Cl. CPC A61K 9/7084; A61K 9/703; A61K 31/381; A61K 47/32; A61K 47/34; A61M 37/00

(57) ABSTRACT

The invention relates to an at least three-layer transdermal therapy system (TTS), which contains, in particular, rotigotine as an active ingredient. The TTS comprises an active ingredient-containing central layer, which comprises at least one hygroscopic polymer or copolymer, an inner, active ingredient-permeable layer and an outer layer.

The invention relates to an at least three-layer transdermal therapy system (TTS), particularly having a decreased cold flow. The invention further particularly relates to a TTS with rotigotine.

Active ingredient-containing patches have already been introduced to the market under the name “transdermal therapy systems” (TTS) in the pharmaceutical therapy of various diseases. The advantages of this form of substance delivery are primarily the extended application intervals, which lead to an improved patient compliance, the avoidance of first-pass metabolism (premature substance breakdown with oral administration) and the pharmacokinetically optimized plasma concentration time profile, which warrants a longer duration of the active ingredient with less adverse events.

TTS have been introduced into therapy, for example, for estradiol, norethisterone acetate, nicotine, fentanyl, tulobuterol, rivastigmine, rotigotine, ethinyl estradiol/norelgestromin, buprenorphine and nitroglycerin as well as an increasing number of further active ingredients. Known TTS often have an active ingredient-impermeable back layer (so called backing), an active ingredient-containing reservoir layer with control membrane and adhesive layer—or one or more matrix layers, optionally with an adhesive layer for attachment to the skin—and an active ingredient-impermeable protective foil (so called release liner) to be removed prior to application to the skin.

Aside from polymers, resins and other pharmaceutical excipients, system components, which are liquid at ambient temperature, are also used to improve permeation of active ingredient through the skin, which components partially serve to adjust the adhesive force, the improvement of diffusion within the TTS or, alternatively, the improvement of permeation of active ingredient through the skin.

An essential goal of optimizing TTS is to improve cohesion of the adhesive layers, in particular to minimize the so called cold flow because non-cohesive systems—which smudge on the skin—become unattractive fast and adhere unreliably. This problematic is increased by the ordinarily emollient effect of the active ingredient.

The phenomenon of cold flow means slow leaking of the viscid adhesive from the edges of the TTS under slight ambient pressure. This phenomenon is extremely undesired because it can cause adherence of the TTS in the respective packaging, even after a few months of storage. In those cases it is often no longer possible to take the TTS out of the packaging with the result that it can no longer be used.

To optimize adhesive compositions, primarily with respect to a decrease of cold flow, numerous efforts have been taken in the past, none of which, however, is satisfactory, yet, because they are partially associated with yet other disadvantages. For example, the increase of cohesion of adhesive compositions by means of chemical cross-linking is associated with the disadvantage of decreased inflow and thus worse adhering to the texture of the skin.

Furthermore, there have been attempts in the past to improve the problem of cold flow for silicone polymers by adding consistency-improving active ingredients (EP 0524776), by drying the basis polymer to improve absorption capacity for the active ingredient (EP 2308480) or by admixing hydrophilic polymers as excipients to the adhesive polymer (WO 2005/099676). To date, none of these solutions has led to any decisively better operational systems.

The identification of suitable strategies to reduce cold flow presents a specific challenge because a corresponding implementation not only has to be compatible with other TTS components and the patch structure, but must also meet the high regulatory requirements (e.g., EMEA Transdermal Guidline). Particularly, the interaction with the active ingredient and the substance delivery is critical, in doing so.

Furthermore, multi-layered TTS are known to the prior art, which cannot reliably solve the problem of cold flow, either. For example, in the TTS according to U.S. Pat. No. 5,004,610, a membrane with better tensile strength is arranged between a softly plasticized reservoir and an adhesive layer, to achieve a membrane-controlled release. Corresponding TTS however, show a distinct cold flow.

A TTS with up to five active ingredient-containing layers is known from U.S. Pat. No. 4,769,028, wherein specific release properties are to be achieved via a substance concentration gradient for nitroglycerin. These layers are uniformly viscid layers, by means of which an improvement of cohesion or decrease of cold flow is not to be expected because it is known that such adhesives dramatically lose mechanical strength with increasing thickness of the total layer without any further strengthening measures.

This applies correspondingly to DE 10 2006 026 060 A1, which discloses a TTS with up to six layers.

Therefore, it is the object of the present invention to provide a TTS, which has at least one improvement with respect to the disadvantages of the TTS known in the art. This object is solved by means of a transdermal therapy system pursuant to claim 1.

Advantageous implementations are the subject matter of corresponding dependant claims.

The TTS according to the invention is characterized in that it comprises
(i) a central layer, which contains at least one active ingredient,
(ii) an inner layer, which is permeable for the at least one active ingredient and
(iii) an outer layer
   as well as optionally a backing and/or a protective foil, wherein the central layer contains at least one hygroscopic polymer or copolymer.

The invention is based on the realization that the use of a hygroscopic polymer or copolymer in the central, active ingredient-containing layer can significantly reduce the problem of cold flow.

Furthermore, it turned out that the use of a hygroscopic polymer or copolymer in the active ingredient-containing layer possesses a positive influence on release of active ingredient. It is assumed that the ambient hygroscopic polymers/copolymers (e.g., from the air or the skin) absorb moisture causing the water balance in the central layer to increase. In doing so, the solubility of active ingredients with limited water solubility, such as rotigotine, decreases in the active ingredient-containing layer, which “squeezes” the active ingredient out of this layer. In doing so, the release of active ingredient is ultimately improved.

This transport process is possibly enhanced by the effect that the diffusion resistance of the hygroscopic phase is lowered by means of water absorption and in doing so, the active ingredient, in particular rotigotine, can easier diffuse from the hygroscopic layer.

In doing so, it is notable that such improvement in release of active ingredient preferably begins particularly at the point in time of application on the patient, therefore application-specifically “activating” the patch. In praxis, this means that only after removal of the TTS from the packaging and application to the skin, can the hygroscopic polymer increase the water balance in the central layer and thus also improve release of active ingredient from the patch by means of moisture absorption from the air and the skin.

By means of the composition of the TTS according to the invention, lower amounts of active ingredient can remain in the patch after application, compared to conventional patches. This increases drug safety and also reduces manufacturing costs of such systems.

Furthermore, it turned out that the patch according to the invention maintains its structural integrity despite significant water absorption in the central layer.

The term “rotigotine” 5,6,7,8-tetrahydro-6-[propyl-[2-(2-thienyl)ethyl]amino]-1-naphthalenol (INN: rotigotine) in terms of the present invention comprises—unless further differentiated—both the free base and the protonated form, that is rotigotine salts, in particular pharmaceutically acceptable salts, such as, e.g., rotigotine hydrochloride. So called prodrugs of rotigotine, which are only converted to an active ingredient in the human organism, are comprised as well.

Rotigotine can be present in various isomeric forms. Accordingly, this term also comprises the isomers or mixtures thereof. Therefore, the S or R enantiomer or the racemate or any other enantiomer mixture of rotigotine can be used.

A “hygroscopic (co)polymer” in terms of the invention is defined as a (co)polymer, which can reversibly bind water molecules under normal conditions (75% rel. humidity, 25° C. and 1013.25 hPa) and can preferably release them at an increase in temperature and/or under vacuum conditions, as well. Conversely, the resulting dehydrated (co)polymer is capable to form water molecules, again. Under normal conditions (25° C. and 1013.25 hPa), the vapor pressure of the hydrated hygroscopic (co)polymers is less than 23 hPa, preferably less than 20 hPa, particularly preferred less than 15 hPa and particularly less than 10 hPa. In a particularly preferred embodiment, the vapor pressure of the hydrated hygroscopic (co)polymers is between 2 and 20 hPa, and particularly between 5 and 15 hPa. The dehydrated hygroscopic (co)polymer possesses the capacity to bind at least 0.05 g, preferably at least 0.10 g, and particularly preferred at least 0.15 g water per gram of the hygroscopic (co)polymer. In a particularly preferred embodiment, the dehydrated hygroscopic (co)polymer can bind up to 10 g, preferably up to 25 g, and particularly preferred up to 35 g per gram hygroscopic (co)polymer.

Polymers or copolymers, however, which consist of more than 50% (w/w) monomer residues, which contain non-polar or uncharged groups, are defined as “hydrophobic”.

The terms “in dissolved form” or “dissolved” in the present invention are to be understood in such way that the active ingredient, which is rotigotine in particular, is present as a homogenous one-phase mixture in the (polymer) matrix of the respective layer. This does not exclude that the dissolved active ingredient (in particular rotigotine) is above the saturation point in its concentration, so that aside from the dissolved active ingredient, non-dissolved active ingredient is also present, be that in its amorphous or crystalline form.

In one particular embodiment, the active ingredient, which is in particular rotigotine, is not present as a component of so called micro-reservoirs. “Micro-reservoirs” are compartments, which are spatially and functionally separated from each other. They can consist of pure active ingredient or a mixture of active ingredient and crystallization inhibitor, which are dispersed in a self-adhesive (polymer) matrix. Micro-reservoirs containing rotigotine are described in EP 1524975 B9.

In the present invention, the term “homogenous” in terms of homogenous mixtures is to be understood as a mixture, wherein pure substances are present, which are mixed on the molecular level, meaning they are a single-phase. Heterogeneous mixtures, such as dispersions, wherein the pure substances are not completely mixed but are present in definitive phases, meaning they are multiple-phase, are to be distinguished thereof. To describe homogeneity, the light microscopic ascertainability must be taken into account. Accordingly, “homogeneously distributed” means that the active ingredient (in particular rotigotine) is essentially present not in form of active ingredient-containing particles or micro-reservoirs or crystals, but dissolved. In particular, homogeneous means the absence of micro-reservoirs with active ingredient.

“Permeable” in terms of the present invention is a layer, which is permeable for the active ingredient (in particular the free base of rotigotine) under application conditions, meaning under conditions on patient's skin.

The term “rotigotine” 5,6,7,8-tetrahydro-6-[propyl-[2-(2-thienyl)ethyl]amino]-1-naphthalenol (INN: rotigotine) in terms of the present invention comprises—unless further differentiated—both the free base and the protonated form, that is rotigotine salts, in particular pharmaceutically acceptable salts, such as, e.g., rotigotine hydrochloride. So called prodrugs of rotigotine, which are only converted to an active ingredient in the human organism, are comprised as well.

Rotigotine can be present in various isomeric forms. Accordingly, this term also comprises the isomers and mixtures thereof. Therefore, the S or R enantiomer or the racemate or any other enantiomer mixture of rotigotine can be used.

In a further embodiment of the invention, the at least one hygroscopic polymer or copolymer in the TTS according to the invention has a water absorption of at least 15 wt.-%, preferably at least 25 wt.-% and particularly preferred of at least 35 wt.-% based on its own weight at 75% relative humidity, 25° C. and 1013.25 hPa after saturation.

In a preferred embodiment, the at least one hygroscopic polymer or copolymer in the TTS according to the invention has a weight proportion of 50% or more, preferably of 60, 70 or 80% or more, particularly preferred of 90% or more, and in particular of 100%, based on the weight of the total polymer of the central layer.

In one embodiment of the invention, the at least one hygroscopic polymer or copolymer is selected from the group consisting of polyvinylpyrrolidone, polyvinyl alcohol, partially hydrolyzed polyvinyl acetate, poly(vinylpyrrolidone-co-vinyl alcohol) and a polysaccharide, preferably a water-soluble starch derivative, a water-soluble cellulose derivate, pullulan and alginate, or a mixture thereof.

In a preferred embodiment of the invention, the at least one hygroscopic polymer is polyvinylpyrrolidone or polyvinyl alcohol.

In a particular embodiment, the inner and/or the outer layer in the TTS according to the invention contains at least a hydrophobic polymer, preferably a mixture of various polyisobutylenes and/or a silicone polymer or a mixture of various silicone polymers.

The hydrophobic polymers cause that the active ingredient, which is squeezed out of the hydrated central layer, cannot accumulate in the inner and/or outer layer, but can instead reach the skin directly through the respective layers.

In a preferred embodiment of the invention, the layers (ii) and (iii) contain identical polymers. Layer (ii) corresponds to the inner and layer (iii) to the outer layer. A TTS thus structured can be manufactured easily and has a bilateral symmetrical concentration gradient with respect to the at least one active ingredient.

Because a thermodynamic solution equilibrium sets in between the three layers, release of active ingredient is better predictable for such a structure with reduced complexity.

Furthermore, the reduction of the basis weight per layer and an extension of the layers to at least three layers can further reduce the problem of unwanted cold flow. In a further embodiment, the amount of layers of the TTS can be more than three, more than 5, more than 6 or also more than 7 and more layers. These more than triple-fold layers preferably have a basis weight of not more than 100 g/m², as well. With a complex TTS constructed with multiple layers, it can become possible to realize complex release profiles of the active ingredient contained in the TTS. In a particular embodiment, two or more various active ingredients can be contained in the TTS. These are preferably present in various layers. It is also conceivable—e.g., for strong analgesics such as opioids and opiates—that one of the two layers contains an antagonist or destruction agent, which does not permeate through the skin but neutralizes the effect of the analgesic by, e.g., receptor blockage or destruction during an extraction attempt or other improper use.

Aside from the described advantages with respect to improved cohesion, reduced cold flow and complex release profiles, the multiple-layer structure offers the possibility to administer more than one active ingredient, including individually adjusted release profiles, respectively. With a combination of a quickly releasing (and, in doing so, quickly depleting) layer with a slower releasing (and, in doing so, longer lasting) layer, it is possible to create a long-lasting therapeutic agent, which has neither a particularly long lag time nor premature depletion. Furthermore, layer combinations can be realized, which realize a burst-like additional increase of the plasma levels of the active ingredient after a specific wear time—helpful, e.g., during the early morning of patients' therapy.

The TTS according to the invention comprises an outer layer (“outer layer” or “layer (iii)”). The outer layer in this context is such a layer, which directly or indirectly attaches to the central active ingredient-containing layer on the side, which is averted from the skin.

The outer layer can be preferably arranged between the central layer and the backing. Pursuant to one embodiment, the outer layer is essentially impermeable for the active ingredient (in particular for rotigotine). It can be self-adhesive or non-self-adhesive. If it is non-self-adhesive, adhesives can be provided to adhesively connect the outer layer with the backing. Pursuant to a different preferred embodiment, the outer layer is identical to the inner layer with respect to its polymeric composition. In this case, the outer layer is also permeable for the active ingredient and in particular for rotigotine.

In a preferred embodiment the TTS according to the invention comprises at least

i) a central layer with a basis weight not exceeding 70 g/m², preferably not exceeding 45 g/m², and particularly preferred not exceeding 30 g/m²
ii) an inner layer (1) with a basis weight not exceeding 70 g/m², preferably not exceeding 45 g/m², and particularly preferred not exceeding 20 g/m², as well as
iii) an outer layer (3) with a basis weight not exceeding 70 g/m², preferably not exceeding 45 g/m², particularly preferred not exceeding 25 g/m², and most particularly preferred not exceeding 10 g/m².

In a particularly preferred embodiment, the basis weight of none of the layers is more than 45 g/m² and in particular more than 30 g/m².

Preferably, the TTS according to the invention consists of exactly three layers, which have the above mentioned preferred basis weights.

The following combinations of basis weights (BW) of the central, inner, and outer layer are specifically preferred (other combinations, however, are also possible).

TABLE 1

| | BW (central layer) | BW (inner layer) | BW (outer layer) |
|---|---|---|---|
| A1. | not exceeding 70 g/m² | not exceeding | not exceeding 70 g/m² |
| | | 70 g/m² | |
| A2. | not exceeding 70 g/m² | not exceeding | not exceeding 45 g/m² |
| | | 70 g/m² | |
| A3. | not exceeding 70 g/m² | not exceeding | not exceeding 70 g/m² |
| | | 45 g/m² | |
| A4. | not exceeding 70 g/m² | not exceeding | not exceeding 45 g/m² |
| | | 45 g/m² | |
| A5. | not exceeding 70 g/m² | not exceeding | not exceeding 10 g/m² |
| | | 45 g/m² | |
| A6. | not exceeding 70 g/m² | not exceeding | not exceeding 45 g/m² |
| | | 20 g/m² | |
| A7. | not exceeding 70 g/m² | not exceeding | not exceeding 10 g/m² |
| | | 20 g/m² | |
| A8. | not exceeding 70 g/m² | not exceeding | not exceeding 10 g/m² |
| | | 70 g/m² | |
| A9. | not exceeding 70 g/m² | not exceeding | not exceeding 70 g/m² |
| | | 20 g/m² | |
| A10. | not exceeding 45 g/m² | not exceeding | not exceeding 70 g/m² |
| | | 70 g/m² | |
| A11. | not exceeding 45 g/m² | not exceeding | not exceeding 45 g/m² |
| | | 70 g/m² | |
| A12. | not exceeding 45 g/m² | not exceeding | not exceeding 70 g/m² |
| | | 45 g/m² | |
| A13. | not exceeding 45 g/m² | not exceeding | not exceeding 45 g/m² |
| | | 45 g/m² | |
| A14. | not exceeding 45 g/m² | not exceeding | not exceeding 10 g/m² |
| | | 45 g/m² | |
| A15. | not exceeding 45 g/m² | not exceeding | not exceeding 45 g/m² |
| | | 20 g/m² | |
| A16. | not exceeding 45 g/m² | not exceeding | not exceeding 10 g/m² |
| | | 20 g/m² | |
| A17. | not exceeding 45 g/m² | not exceeding | not exceeding 10 g/m² |
| | | 70 g/m² | |
| A18. | not exceeding 45 g/m² | not exceeding | not exceeding 70 g/m² |
| | | 20 g/m² | |
| A19. | not exceeding 30 g/m² | not exceeding | not exceeding 70 g/m² |
| | | 70 g/m² | |
| A20. | not exceeding 30 g/m² | not exceeding | not exceeding 45 g/m² |
| | | 70 g/m² | |
| A21. | not exceeding 30 g/m² | not exceeding | not exceeding 70 g/m² |
| | | 45 g/m² | |
| A22. | not exceeding 30 g/m² | not exceeding | not exceeding 45 g/m² |
| | | 45 g/m² | |
| A23. | not exceeding 30 g/m² | not exceeding | not exceeding 10 g/m² |
| | | 45 g/m² | |
| A24. | not exceeding 30 g/m² | not exceeding | not exceeding 45 g/m² |
| | | 20 g/m² | |
| A25. | not exceeding 30 g/m² | not exceeding | not exceeding 10 g/m² |
| | | 20 g/m² | |
| A26. | not exceeding 30 g/m² | not exceeding | not exceeding 10 g/m² |
| | | 70 g/m² | |
| A27. | not exceeding 30 g/m² | not exceeding | not exceeding 70 g/m² |
| | | 20 g/m² | |

In respectively preferred embodiments, the TTS comprises exactly a three-layer structure according to one of the examples A1 to A36 stated in the table above. Preferably, the TTS can consist of these three layers, optionally in addition of a backing and a protective layer.

The TTS according to the invention preferably has a total basis weight not exceeding 210 g/m², preferably not exceeding 135 g/m², further preferred not exceeding 75 g/m², and particularly preferred not exceeding 60 g/m².

The basis weights of the active ingredient-containing central layer and the inner layer are preferably in a ratio of 5:1 to 1:5, further preferred of 3:1 to 1:3, and particularly preferred of 3:2 or 1:1.

The basis weights of the inner and the outer layer are preferably in a ratio of 5:1 to 1:5, further preferred of 4:1 to 1:2, and particularly preferred of 2:1.

The basis weights of the central and the outer layer are preferably in a ratio of 5:1 to 1:5, further preferred of 4:1 to 1:2, and particularly preferred of 3:1.

In one embodiment, the basis weights of the outer, the central, and the inner layer are in a ratio of 1:1:1.

In another embodiment, the basis weights are in a ratio of 2:3:2.

In another preferred embodiment, the basis weights are in a ratio of 1:3:2.

Preferably, the central layer is arranged as separation layer, i.e., it has a tensile strength of at least 0.1 N/mm², preferably of at least 2 N/mm², and particularly preferred of 10 N/mm².

The tensile strength of the separation layers as well as the adhesive layers can be measured with established tensile test machines (e.g., PrecisionLine test machines of the company Zwick-Roell). To do so, it is first required to create active ingredient-free solutions of the base material (see below) because otherwise manufacturing-technical differences in the admixing of the active ingredients to individual layers present an incorrect picture. After the preparative creation of films by means of a layering process pertinent to the person skilled in the art by using solvents for the respective base material, cuttings of equal size of these films are clamped into the corresponding test apparatuses. Then, the characteristic force displacement curves are noted. For purposes of uniformity of terminology, the tensile strength (the measured tensile strength value) is measured upon reaching a 10 percent elongation of the base material, respectively.

The measuring of the tensile strengths is standardized to the same layer thickness for purposes of uniformity and obtaining of material.

The adhesive force is defined as the peel force at 900 to steel (also referred to below as: adhesive force (to steel)).

Preferably, the inner layer is arranged as an adhesive layer, i.e., it has an adhesive force (to steel) of at least 1 N/cm², preferably of at least 5 N/cm², and particularly preferred of at least 10 N/cm².

Preferably, the outer layer is arranged as an adhesive layer, i.e., it has an adhesive force (to steel) of at least 1 N/cm^{z}, preferably of at least 5 N/cm², and particularly preferred of at least 10 N/cm².

In a further embodiment, both the outer layer and the inner layer are arranged as an adhesive layer, i.e., they both have an adhesive force (to steel) of at least 1 N/cm², preferably of at least 5 N/cm², and particularly preferred of at least 10 N/cm².

In a preferred embodiment, the central layer is arranged as a separation layer and the inner and the outer layer are arranged as an adhesive layer, respectively.

In a further embodiment, the TTS according to the invention comprises at least two, preferably at least three double layers and optionally a further adhesive layer, wherein the double layers consist of a separation layer and an adhesive layer, respectively.

One separation layer and one adhesive layer, respectively can (sic: missing verb in source text; probably: form) a double layer. Double layer in terms of the invention means that these layers are directly consecutive.

In a preferred embodiment, the transdermal therapy system (TTS) according to the invention comprises at least two such double layers and optionally a protective foil and/or backing. In this embodiment, the TTS according to the invention can comprise a total of at least two separation layers, which provide particular mechanical stability to the TTS, as well as at least two separate adhesive layers. The separation of the adhesive layer of the TTS into at least two adhesive layers and the combinations thereof with the at least two separation layers can, once again, improve the decrease of undesired cold flow.

Furthermore, a structure of the TTS according to the invention with at least two double layers can further facilitate adjusting of specific and complex profiles of release of active ingredient or facilitate such adjusting in the first place.

In an embodiment according to the invention, the separation layer and the adhesive layer differ in their respective tensile strength. In doing so, the separation layer can have a higher tensile strength than the adhesive layer. Such strength is preferably higher by at least a factor of 2, particularly preferred by at least a factor of 5, even further by at least a factor of 10, or a factor of 20. In a particularly preferred embodiment, the tensile strength of the separation layer is higher than the tensile strength of the adhesive layer by at least a factor of 30.

The at least two double layers of the TTS according to the invention pursuant to this embodiment can be identical or different with respect to the tensile strengths of their respective separation or adhesive layers. In doing so, the ratios of the tensile strengths can vary, particularly regarding the adjustment to the respective layer thickness.

In a preferred embodiment, the adhesive layer and the separation layer differ in their respective adhesive force. Preferably, the adhesive layer has a higher adhesive force relative to the respective separation layer by at least a factor of 1,5-2, preferably by at least a factor of 3, particularly preferred by at least a factor of 5.

In a particular embodiment of the double layer, the tensile strength of the adhesive layer does not exceed 0.1 N/mm² and the adhesive force (to steel) of the separation layer does not exceed 0.01 N/cm², preferably not exceeding 0.001 N/cm². The double layer can be present in a TTS according to the invention one-fold or also multiple times (at least two-fold).

In a most particularly preferred embodiment, the adhesive layer has an adhesive force (to steel) of at least 1 N/cm², preferably of at least 10 N/cm², and a tensile strength not exceeding 0.1 N/mm² as well as a basis weight not exceeding 70 g/m², preferably not exceeding 45 g/m², particularly preferred not exceeding 25 g/m². In this embodiment, the separation layer has a tensile strength of more than 0.1 N/mm², preferably of more than 2 N/mm², and an adhesive force (to steel) not exceeding 0.01 N/cm², preferably not exceeding 0.001 N/cm² as well as a basis weight not exceeding 30 g/m², preferably not exceeding 20 g/m², particularly preferred not exceeding 10 g/m².

The TTS according to the invention can have at least two, preferably at least three, further preferred at least four double layers with one separation layer and one adhesive layer, respectively, as well as optionally a further adhesive layer. The double layers can be identical or different with respect to the properties adhesive force, tensile strength and basis weight. Preferred are at least two double layers, which are identical with respect to these properties.

The at least two double layers according to this embodiment pursuant to the invention can be directly sequenced so that there is a sequence of separation layer—adhesive layer—separation layer—adhesive layer (or a multiple thereof). In a further embodiment, the respective double layers can also be separated from one another by at least one further layer.

The inner layer—which is one adhesive layer of the double layers in the above described specific embodiments—contains at least one self-adhesive, physiologically tolerable polymer, respectively. Preferred is a base polymer, selected from the group of the following polymers: polyisobutylene (PIB), a mixture of various polyisobutylenes (PIBs), silicone polymer, a mixture of various silicone polymers, acrylate copolymers, acrylic ester copolymers, butyl rubber, polybutylene, styrene copolymers, in particular styrene-butadiene-styrene block copolymers and styrene-iosprene copolymers, in particular styrene-iosprene, in particular styrene-iosprene-styrene block copolymers, ethylene vinylacetate copolymers (EVA), mixtures and copolymers thereof.

Particularly preferred as base polymer of the inner layer are PIB, a mixture of various PIBs, silicone polymer, a mixture of various acrylate copolymers, butyl rubber, polybutylene, styrene-iosprene-styrene block copolymers, styrene-butadiene-styrene block copolymers, EVA, mixtures and copolymers thereof.

Particularly preferred are a PIB, a mixture of various PIBs, a silicone polymer as well as a mixture of various silicone polymers. Most particularly preferred are a PIB as well as a mixture of various PIBs.

Pursuant to one embodiment, a PIB of higher molecular weight is used.

The molecular weight (or mol mass) of the individual molecules of polymers is distributed over a more or less wide area due to the different degrees of polymerization. Therefore, no exact molar masses can be stated for polymers, but only a molar mass distribution (often referred to as MWD, molecular weight distribution). Such distribution describes the distribution for a specific substance, in other words the distribution of the molar mass of the molecules contained. The person skilled in the art knows various average values for the definition of the molecular weight, such as the number-average, the viscosity-average or the weight-average and the determination thereof. Below, the molecular weights of the polymers are defined particularly via the weight-average and the number-average.

In doing so, the PIB of higher molecular weight preferably has a weight-average molecular weight Mw of 100.000 to 1,000,000 g/mol, preferably of 150,000 to 800,000 g/mol, further preferred of 200,000 to 700,000 g/mol, and particularly preferred of 250,000 to 600,000 g/mol.

In a preferable embodiment, the PIB of higher molecular weight preferably has a number-average molecular weight Mw of 100,000 to 1,000,000 g/mol, preferably of 150,000 to 800,000 g/mol, further preferred of 200,000 to 700,000 g/mol, and particularly preferred of 250,000 to 600.000 g/mol.

For example, a PIB with a Mw of about 250,000 g/mol or a PIB with a Mw of about 600,000 g/mol can be used.

Pursuant to other embodiments, a mixture of two PIBs of different molecular weight is used. In doing so, a mixture of a PIB of higher molecular weight with a PIB of lower molecular weight is preferably used.

On doing so, the PIB of lower molecular weight preferably has a weight-average molecular weight Mw of 10,000 to 100,000 g/mol, preferably of 20,000 to 50,000, further preferred of 30,000 to 40,000, and particularly preferred of about 36,000 g/mol.

In doing so, the PIB of lower molecular weight can preferably have a number-average molecular weight Mw of 10.000 to 100.000 g/mol, preferably of 20.000 to 50.000, further preferred of 30.000 to 40.000, and particularly preferred of about 36.000 g/mol.

In one preferred aspect of the invention, a mixture of a PIB with a molecular weight MW of 250,000 to 600,000 g/mol and of a PIB with a lower molecular weight MW of about 36,000 g/mol is used.

Preferably, a low molecular polybutylene is added to this mixture, as well.

PIBs from the Oppanol series by the manufacturer BASF and/or from the Durotak series by the manufacturer Henkel can be used, for example. Oppanol 10, (low molecular polyisobutylene), Oppanol 12 (low molecular polyisobutylene), Oppanol 100 (high molecular polyisobutylene), Oppanol 200, Durotak 87-6908 as well as Durotak 618a are to be named as examples. The PIBs from the Durotak series, however, can easily be mixed by the person skilled in the art him-/herself, e.g., from those of the Oppanol series, such as B100, B10, etc.:

The DUROTAK adhesive DT-618A can be mixed as follows, for example:

DT-618A: 12.5% Oppanol B 100+62.5% Oppanol B 10+25% polybutene 950.

Further PIBs according to the invention can be mixed as follows:

DT-618A modified: 24% Oppanol B 100+51% Oppanol B 10+25% polybutene 950 DK: 23.4% Oppanol B 100+33.5% Oppanol B12+Indopol L14 (polybutene) 43%.

A preferred PIB adhesive is the DK, which consists of 23.4 wt.-% PIB with a number-average Mw of 600,000 g/mol, 33.5 wt.-% PIB with a weight-average Mw of about 51,000 g/mol and 43 wt.-% polybutene of a polybutene with a number-average Mw of about 370 g/mol.

Preferably, the silicone polymers used in the inner layer of the TTS are of the type that forms a soluble poly-condensed polydimethylsiloxane (PDMS)/resin network, wherein the hydroxyl groups are capped with, e.g., trimethylsilyl (TMS) groups. Preferably, the weight ratio of resin to PDMS is 85:15 to 35:65, preferably 75:25 to 45:55, and particularly preferred 65:35 to 55:45. Preferred silicone polymers of this type are BIO-PSA pressure-sensitive silicone adhesives, manufactured by Dow Corning, in particular 07-420x- and 07-430x qualities, wherein the x presents a manufacturer's number code, which characterizes the solvent used by the respective adhesive (x=1: heptane, x=2: ethylacetate, x=3: toluene). However, other silicone adhesives can also be used. BIO-PSA 07-420x has medium adhesiveness with a resin-PDMS weight ratio of 65:35, BIO-PSA 07-430x, however, has high adhesiveness with a ratio of 55:45.

In a further and particularly preferred aspect, two or more silicone adhesives are used as main adhesive components. It can be advantageous, if such a mixture of silicone adhesives contains a mixture of powerfully adhesive, pressure-sensitive adhesives comprising PDMS with a resin (e.g., 07-430x), and medium-strength-adhesive, pressure-sensitive silicone adhesives comprising PDMS with a resin (e.g., 07-420x).

Such a mixture comprising a pressure-sensitive silicone adhesive with powerful and medium-strength adhesiveness, which comprises PDMS with a resin, is advantageous because it provides an optimal balance between good adhesion and low cold flow. Excess cold flow can result in a patch, which is too soft, which easily sticks to the packaging or patient's clothing. Furthermore, such a mixture can be particularly useful to obtain higher plasma levels. A mixture from the previously mentioned 07-420x (average adhesiveness) and 07-430x (high adhesiveness) proved to be particularly useful in the inner layer for the TTS pursuant to the present invention. In doing so, mixture ratios between silicone adhesives with average adhesiveness and silicone adhesives are preferred, which have high adhesiveness of 1:50 to 50:1, particularly preferred of 1:10 to 10:1, and in particular of about 1:1.

In a further aspect of the invention, silicone polymers with low adhesive force (“low tack”=identifier 440X), average adhesive force (“medium tack”=identifier 450X), or with high adhesive force (“high tack”=identifier 460X) are employed. These are silicone polymers of the type that form a soluble poly-condensed polydimethylsiloxane (PDMS)/resin network. Preferably, the weight ratio of resin to PDMS is 85:15 to 35:65, preferably 75:25 to 45:55, and particularly preferred 65:35 to 55:45. These three classes of silicone adhesives are summarized in table 2 under the term “standard silicone adhesives”. Selected examples are BIO-PSA 7-4401, BIO-PSA 7-4402, BIO-PSA 7-4501, BIO-PSA 7-4502, BIO-PSA 7-4601, and BIO-PSA 7-4602.

In a preferable aspect of the invention, the silicone adhesive BIO-PSA 7-4502 is employed.

In an additional aspect of the invention, amine-compatible silicone adhesives with low adhesive force (“low tack”=identifier 410X), average adhesive force (“medium tack”=identifier 420X) or with high adhesive force (“high tack”=identifier 430X) are employed. These three classes of silicone adhesives are summarized under the term “AK silicone adhesives” in table 2. Selected examples are BIO-PSA 7-4101, BIO-PSA 7-4102, BIO-PSA 7-4201, BIO-PSA 7-4202, BIO-PSA 7-4301, and BIO-PSA 7-4302.

In a preferable aspect of the invention, the amine-compatible silicone adhesive BIO-PSA 7-4202 is employed.

In a further aspect of the invention, the silicone adhesive contains an oil, which is suitable to influence the adhesive properties of the silicone adhesive. The oil can be added to the highly, average, and low adhesive silicone adhesives.

The oil can be selected from silicone oils, paraffin oils, and neutral oils.

Polydimethylsiloxanes with kinematic viscosities in the area of 100 to 12.500 m²·s⁻¹ can be particularly used as silicone oils. Customary products are Dow Corning Q7-9120 polydimethylsiloxanes with a viscosity of 20 m²·s⁻¹, 100 m²·s⁻¹, 1000 m²·s⁻¹, or 12.500 m²·s⁻¹. Neutral oils according to the invention are meant to be mid-chained triglycerides. These are, in particular, triglycerides from caprylic acid and/or capric acid chains. Paraffin oils according to the invention are meant to be white oils of medical quality (paraffinum liquidum). Examples for white oils employable according to the invention are Shell Ondina 933 and 941. Shell Ondina 933 is a white oil with a density of 0.883 g/cm³ at 15° C., a dynamic viscosity of 212 mPas at 20° C., and a molecular weight of 415 g/mol. Shell Ondina 941 is a white oil with a density of 0.868 g/cm³ at 15° C., a dynamic viscosity of 268 mPas at 20° C., and a molecular weight of 530 g/mol.

In a preferable aspect, a silicone oil is used in the silicone layer. Particularly preferred is the use of polydimethylsiloxane with the viscosity 12.500 m²·s⁻¹.

In a further aspect, the concentration of the oil in the silicone adhesive is in the area of 0.01 wt.-% to 10 wt.-% with respect to the silicone adhesive. Below of 0.01 wt.-%, the oil has no effect on the properties of the silicone adhesive. More than 10 wt.-% oil cannot be incorporated into the silicone adhesive. Preferably, the concentration of the oil is in the area of 0.1 wt.-% to 5 wt.-%. Above 5%, if it can be incorporated, the oil can cause a cold flow of the silicone adhesive. Below 0.1 wt.-%, the effect achieved with the oil is insufficient. Particularly preferred, the concentration of the oil is in the area of 0.5 wt.-% to 1.5 wt.-%. In particular, the concentration of oil in the silicone adhesive is about 1%.

In a further aspect of the invention, so called hot melt silicone adhesives are used, which are solvent-free and become liquid by means of heat treatment. These silicone adhesives are described as “HM silicone adhesives” in table 2.

In one embodiment of the invention, styrene block copolymer-based adhesives, which carry non-elastomer styrene blocks at the ends and elastomer blocks in the middle (sic: missing verb in source text; probably: are used). These are described as “SxS pressure-sensitive adhesives” in table 2. The elastomer blocks can consist of polyethylene butylene, polyethylene propylene, polybutadiene, polyisobutylene, or polyisoprene, for example.

Suitable SxS adhesives are described in U.S. Pat. No. 5,559,165 or U.S. Pat. No. 5,527,536 for example, and are characterized by good adhesive properties, simple manufacturing and processing as well as good skin tolerance.

SxS pressure-sensitive adhesives can be obtained both commercially (e.g., as Duro Tak 378-3500 by National Starch & Chemical) and with hot-melt extrusion equipment during the production of the active ingredient-containing patches themselves. To do so, corresponding amounts (at least of the following components) of a styrene block copolymer (e.g., Shell Kraton GX1657 or Kraton D-1107CU) are metered into the extruder with an aliphatic and/or aromatic resin (e.g., Keyser Mackay Regalite R1090 or Regalite R1010 or Regalite R1100) and an oil (e.g., Shell Ondina 933 or Ondina 94) from the individual metering stations, and mixed and melted therein. In the last step, the active ingredient is metered into the pressure-sensitive adhesive thus manufactured and the composition is laminated to foils. Typical exemplary weight proportions of polymer:resin:oil are, e.g., 100:120:20 or 100:200:50. By means of variation of these quantity proportions, the properties of the SxS pressure-sensitive adhesive can be adjusted to the desired properties of the TTS (adhesive force, minimal cold flow, duration of adhesiveness, release profile of the substance, etc.), respectively.

A further embodiment of the invention uses a copolymer of butene and isobutylene. It is described as “BIB” in table 2. An example is PAR 950.

One embodiment uses a thermoplastic polymer of butene-1 as polymer for the inner layer. It is described as “polybutene” in table 2. Contrary to branched-structured polyisobutylene, the monomers in polybutene are arranged linear and mostly isotactic, wherein high molar masses of 700,000 to 3,000,000 g/mol are achieved, overall. An exemplary product is Indopol.

One aspect of the invention uses a copolymer of ethylene and vinylacetate as polymer for the inner layer. It is described as “EVA” in table 2.

It is also possible that the above mentioned hydrophobic polymers and copolymers contain further hydrophilic monomers, wherein the proportion of these hydrophilic monomers does not exceed 50 mol %, preferably not exceeding 30 mol %, particularly preferred not exceeding 10 mol %.

The base polymer of the inner layer is preferably hydrophobic. The adhesive layer contains base polymer of at least 20 wt.-%, preferably at least 30 wt.-%, particularly preferred at least 40 wt.-%. Most particularly preferred, the inner layer contains at least 80 wt.-% base polymer.

The outer layer—that is an adhesive layer of the double layers in the above described embodiments—contains at least a self-adhesive, physiologically tolerable polymer, respectively. Preferred is a base polymer, which is selected from the group of the following polymers: polyisobutylene (PIB), a mixture of various polyisobutylenes, silicone polymer, a mixture of various silicone polymers, acrylate copolymers, acrylic ester copolymers, butyl rubber, polybutylene, styrene copolymers, in particular styrene-butadiene-styrene block copolymers and styrene-iosprene copolymers, in particular styrene-iosprene, in particular styrene-iosprene-styrene block copolymers, ethylene vinylacetate copolymers (EVA), mixtures and copolymers thereof.

Particularly preferred as base polymer of the outer layer are PIB, a mixture of various PIBs, silicone polymer, a mixture of various silicone polymers, acrylate copolymers, butyl rubber, polybutylene, styrene-iosprene-styrene block copolymers, styrene-butadiene-styrene block copolymers, EVA, mixtures and copolymers thereof.

Particular preferred are a PIB, a mixture of various PIBs, a silicone polymer as well as a mixture of various silicone polymers. Most particularly preferred are a PIB as well as a mixture of various PIBs.

The base polymer of the outer layer is preferably hydrophobic. The adhesive layer contains base polymer of at least 20 wt.-%, preferably at least 30 wt.-%, particularly preferred at least 40 wt.-%. Most particularly preferred, the outer layer contains at least 80 wt.-% base polymer.

Furthermore, the above specified embodiments of the inner layer can be identically applied to the inner layer.

As already mentioned, the outer and the inner layer can have different polymer compositions. Preferably, however, the outer layer has the same polymer composition as the inner layer.

As base polymer of the central layer—which corresponds to the separation layer in the above mentioned embodiments of the TTS with a double layer—all physiologically tolerable hygroscopic polymers are suitable, which achieve a layer with the preferred higher tensile strength with respect to the inner and outer layer (adhesive layer). The base polymer of the central layer is preferably selected from the group of the following polymers: polyvinyl alcohol (PVA), polyvinylpyrrolidone (PVP), partially hydrolyzed polyvinyl acetate, poly(vinylpyrrolidone-co-vinyl alcohol), polysaccharides, preferably a water-soluble starch derivative, a modified cellulose, which is preferably water-soluble, pullulan and alginate as well as mixtures and copolymers thereof.

Particularly preferred are PVP, PVA as well as mixtures or copolymers thereof. Most particularly preferred is PVP.

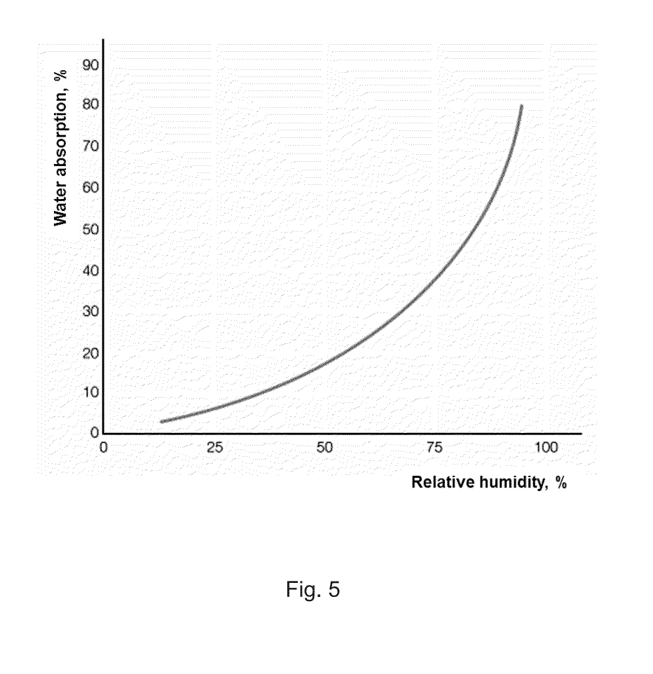

According to the invention, the PVP can be basically used in all available molecular weights. It is known for PVPs that the hygroscopicity profile, as exemplary represented in FIG. 5 for a soluble PVP, is applicable to all PVP types and is thus independent of the molecular weight of the respective PVP. The specific selection of one or more PVPs for the central layer of the TTS according to the invention is well familiar to the person skilled in the art and the embodiments taught below are thus only intended to be illustrative do not present any limitation of the invention.

Preferably, PVP with a weight-average molecular weight Mw of 20,000 to 3,000,000 g/mol, preferably of 100,000 to 2,500,000 g/mol, further preferred of 500,000 to 2,000,000 g/mol, and particularly preferred of 1,000,000 to 1,500,000 g/mol is used.

In one embodiment of the invention, a soluble polyvinylpyrrolidone derivate is employed, which is described as “sol. PVP” in table 2. Preferable examples for sol. PVAs comprise Kollidon 12 PF, Kollidon 17 PF, Kollidon 25, Kollidon 30, Kollidon 30 LP, and Kollidon 90 F.

In a further embodiment of the invention, a non-soluble, cross-linked polyvinylpyrrolidone derivate is employed, which is described as “CL PVP” in table 2.

Preferable examples for CL PVAs comprise Kollidon CL, Kollidon CL-F, Kollidon CL-SF, and Kollidon CL-M.

In an additional embodiment of the invention, a copolymer of 1-vinyl-2-pyrrolidone and vinylacetate is employed, preferably in a mass ratio of 6:4, which is described as “VPNac” in table 2. Preferable examples for VPNAc comprise Kollidon VA64 and Kollidon VA64 Fine.

For the PVA used according to the invention, PVAs in all available molecular weights, hydrolysis and polymerization degrees can basically be used. The specific selection of one or more PVAs for the central layer of the TTS according to the invention is familiar to the person skilled in the art and the embodiments taught below are only intended to be illustrative and do not present any limitation of the invention.

Preferably, PVA is used with a weight-average molecular weight MW of 5,000 to 100,000 g/mol, preferably of 10,000 to 50,000 g/mol, further preferred of 20,000 to 40,000 g/mol, and particularly preferred of about 31,000 g/mol.

A preferable PVA has a number-average molecular weight Mw of 5,000 to 100,000 g/mol, preferably of 10,000 to 50,000 g/mol, further preferred of 20,000 to 40,000 g/mol, and particularly preferred of about 31,000 g/mol.

The average degree of polymerization Pw of the employed PVA is preferably between 100 to 2050, preferably between 200 to 1025, further preferred between 400 to 825, and particularly preferred around 630.

The degree of hydrolysis (saponification) of the PVA is preferably 75 to 100 mol %, preferably 80 to 95 mol %, further preferred 85 to 90 mol %, and particularly preferred about 87±1 mol %.

In one embodiment of the invention, partially hydrolyzed polyvinyl alcohol is employed, which is described as “PH PVA” in table 2. Preferable examples for PH PVAs are Mowiol 3-85, Mowiol 4-88, Mowiol 5-88, Mowiol 8-88, Mowiol 13-88, Mowiol 18-88, Mowiol 23-88, Mowiol 26-88, Mowiol 32-88, Mowiol 40-88, Mowiol 47-88, and Mowiol 30-92.

In an alternative embodiment of the invention, completely hydrolyzed polyvinyl alcohol is employed, which is described as “CH PVA” in table 2. Preferable examples for CH PVAs are Mowiol 4-98, Mowiol 6-98, Mowiol 10-98, Mowiol 20-98, Mowiol 30-98, Mowiol 56-98, Mowiol 15-99, and Mowiol 28-99.

PVP of the Kollidon series from the manufacturer BASF, in particular Kollidon 90 F, as well as PVA of the Mowiol series from the manufacturer Clariant, in particular Mowiol 4-88, can be used, for example.

For the polysaccharides used according to the invention, polysaccharides in all available molecular weights, degrees of branching and substitution patterns can be basically employed. The specific selection of one or more polysaccharides for the central layer of the TTS is familiar to the person skilled in the art and the embodiments taught below are only intended to be illustrative and do not present any limitation of the invention. For a layer with a particularly strong hydroscopicity, the person skilled in the art can conveniently use conjac gum, which has an extremely high water absorption capacity of 1:50.

Pursuant to the invention, “polysaccharides” are meant to be molecules, wherein at least 10 monosaccharide molecules are bound via a glycosidic bond. Preferred examples comprise alginates, agar-agar, carrageen, guar gum, conjac gum, carob bean gum, oat beta-glucan, pectin, xanthan, guar hydroxypropyltrimonium chloride and sodium hyaluronate.

For the modified celluloses used according to the invention, cellulose derivatives in all available molecular weights, degrees of branching and substitution patterns can basically be employed. The specific selection of one or more modified celluloses for the central layer of the TTS according to the invention is familiar to the person skilled in the art and the embodiments taught below are thus only intended to be illustrative and do not present any limitation of the invention.

The modified celluloses are described as “mod. celluloses” in table 2. Preferable examples are Ethylcellulose (EC), MC (Metolose®, Methylcellulose, Cellulose-methylated), HPMC (Metolose®, MHPC, Hypromellose, Hydroxypropyl Methylcellulose), HPMC-Phthalat (HPMC-P, Hypromellose-Phthalate), AQOAT (HPMC-AS, Hypromellose-Acetate-Succinate), L-HPC (Hydroxypropyl Cellulose, low-substituted), USP, Carboxy Methylcellulose (CMC) and Microcrystalline Cellulose (MCC).

It is also possible that the above mentioned hydrophilic polymers and copolymers contain further hydrophobic monomers, wherein the proportion of these hydrophobic monomers does not exceed 50 mol %, preferably not exceeding 30 mol %, particularly preferred not exceeding 10 mol %.

The at least one base polymer used in the central layer is preferably hydrophilic. It is furthermore also non-self-adhesive. The central layer preferably contains base polymer of at least 20 wt.-%, preferably at least 30 wt.-%, most particularly preferred at least 40 wt.-%.

Advantageous combination of polymers of the central and the inner/outer layer are listed in the following table 2.

According to the invention, all polymers of the central layer listed above can be combined with all polymers of the inner layer, listed above. The preferred combination possibilities taught below are therefore only intended to be illustrative and do not present any limitation of the invention. It is subject to the expert knowledge of the person skilled in the art having knowledge of the invention to identify suitable polymers and the combinations thereof.

TABLE 2

| Central layer | Inner and outer layer |
|---|---|
| PH PVA | Standard silicone adhesive |
| CH PVA | Standard silicone adhesive |
| sol. PVP | Standard silicone adhesive |
| CL PVP | Standard silicone adhesive |
| Vp/VAc | Standard silicone adhesive |
| polysaccharides | Standard silicone adhesive |
| mod. celluloses | Standard silicone adhesive |
| PHPVA | AK silicone adhesive |
| CH PVA | AK silicone adhesive |
| sol. PVP | AK silicone adhesive |
| CL PVP | AK silicone adhesive |
| Vp/VAc | AK silicone adhesive |
| polysaccharides | AK silicone adhesive |
| mod. celluloses | AK silicone adhesive |
| PH PVA | SxS pressure-sensitive |
| | adhesive |
| CH PVA | SxS pressure-sensitive |
| | adhesive |
| sol. PVP | SxS pressure-sensitive |
| | adhesive |
| CL PVP | SxS pressure-sensitive |
| | adhesive |
| Vp/VAc | SxS pressure-sensitive |
| | adhesive |
| polysaccharides | SxS pressure-sensitive |
| | adhesive |
| mod. celluloses | SxS pressure-sensitive |
| | adhesive |
| PH PVA | PIB |
| CH PVA | PIB |
| sol. PVP | PIB |
| CL PVP | PIB |
| Vp/VAc | PIB |
| polysaccharides | PIB |
| mod. celluloses | PIB |
| PH PVA | polybutene |
| CH PVA | polybutene |
| sol. PVP | polybutene |
| CL PVP | polybutene |
| Vp/VAc | polybutene |
| polysaccharides | polybutene |
| mod. celluloses | polybutene |
| PH PVA | EVA |
| CH PVA | EVA |
| sol. PVP | EVA |
| CL PVP | EVA |
| Vp/VAc | EVA |
| polysaccharides | EVA |
| mod. celluloses | EVA |
| PH PVA | BIB |
| CH PVA | BIB |
| sol. PVP | BIB |
| CL PVP | BIB |
| Vp/VAc | BIB |
| polysaccharides | BIB |
| mod. celluloses | BIB |

According to the invention, TTS with one of the combinations of base polymers in the respective layers listed in table 3 are particularly preferred:

The following definitions are denoted in table 3:

“PIB”: Mixture of a PIB with higher molecular weight, in particular Mw=250,000 to 600,000 g/mol, and a PIB with lower molecular weight, in particular Mw=about 36,000 g/mol, and preferably a low-molecular polybutylene

“silicone polymer”: silicone polymer, in particular a poly-condensed PDMS/resin network with a resin-PDMS weight ratio of 65:35 to 55:45

“silicone polymer mixture”: Mixture of two silicone polymers, in particular two poly-condensed PDMS/resin networks, one with a resin-PDMS weight ratio of about 65:35 and one with a ratio of about 55:45

“styrene-iosprene”: styrene-iosprene copolymers, in particular styrene-iosprene-styrene block copolymer

“styrene-butadiene”: styrene-butadiene-styrene block copolymer

“PVA”: PVA, in particular with Mw=about 31,000 g/mol, Pw=about 630, hydrolysis degree=about 87±1 mol %

“PVP”: PVP, in particular with Mw=1,000,000 to 1,500,000 g/mol

“polyvinyl acetate, p.h.”=partially hydrolyzed polyvinyl acetate

“water-soluble starch derivative”=water-soluble starch derivative

“water-soluble cellulose derivative”=water-soluble cellulose derivative

TABLE 3

| | inner layer | central layer | outer layer |
|---|---|---|---|
| B1. | PIB | poly(vinylpyrrolidone- | PIB |
| | | co-vinyl alcohol | |
| B2. | PIB | poly(vinylpyrrolidone- | silicone polymer |
| | | co-vinyl alcohol | |
| B3. | PIB | poly(vinylpyrrolidone- | silicone polymer mixture |
| | | co-vinyl alcohol | |
| B4. | PIB | poly(vinylpyrrolidone- | acrylate copolymer |
| | | co-vinyl alcohol | |
| B5. | PIB | poly(vinylpyrrolidone- | acrylic ester copolymer |
| | | co-vinyl alcohol | |
| B6. | PIB | poly(vinylpyrrolidone- | butyl rubber |
| | | co-vinyl alcohol | |
| B7. | PIB | poly(vinylpyrrolidone- | PIB/polybutylene |
| | | co-vinyl alcohol | |
| B8. | PIB | poly(vinylpyrrolidone- | styrene-iosprene |
| | | co-vinyl alcohol | |
| B9. | PIB | poly(vinylpyrrolidone- | styrene-butadiene |
| | | co-vinyl alcohol | |
| B10. | PIB | alginate | PIB |
| B11. | PIB | alginate | silicone polymer |
| B12. | PIB | alginate | silicone polymer mixture |
| B13. | PIB | alginate | acrylate copolymer |
| B14. | PIB | alginate | acrylic ester copolymer |
| B15. | PIB | alginate | butyl rubber |
| B16. | PIB | alginate | PIB/polybutylene |
| B17. | PIB | alginate | styrene-iosprene |
| B18. | PIB | alginate | styrene-butadiene |
| B19. | PIB | PVA | PIB |
| B20. | PIB | PVA | silicone polymer |
| B21. | PIB | PVA | silicone polymer mixture |
| B22. | PIB | PVA | acrylate copolymer |
| B23. | PIB | PVA | acrylic ester copolymer |
| B24. | PIB | PVA | butyl rubber |
| B25. | PIB | PVA | PIB/polybutylene |
| B26. | PIB | PVA | styrene-iosprene |
| B27. | PIB | PVA | styrene-butadiene |
| B28. | PIB | PVP | PIB |
| B29. | PIB | PVP | silicone polymer |
| B30. | PIB | PVP | silicone polymer mixture |
| B31. | PIB | PVP | acrylate copolymer |
| B32. | PIB | PVP | acrylic ester copolymer |
| B33. | PIB | PVP | butyl rubber |
| B34. | PIB | PVP | PIB/polybutylene |
| B35. | PIB | PVP | styrene-iosprene |
| B36. | PIB | PVP | styrene-butadiene |
| B37. | PIB | water-soluble starch | PIB |
| | | derivative | |
| B38. | PIB | water-soluble starch | silicone polymer |
| | | derivative | |
| B39. | PIB | water-soluble starch | silicone polymer mixture |
| | | derivative | |
| B40. | PIB | water-soluble starch | acrylate copolymer |
| | | derivative | |
| B41. | PIB | water-soluble starch | acrylic ester copolymer |
| | | derivative | |
| B42. | PIB | water-soluble starch | butyl rubber |
| | | derivative | |
| B43. | PIB | water-soluble starch | PIB/polybutylene |
| | | derivative | |
| B44. | PIB | water-soluble starch | styrene-iosprene |
| | | derivative | |
| B45. | PIB | water-soluble starch | styrene-butadiene |
| | | derivative | |
| B46. | PIB | pullulan | PIB |
| B47. | PIB | pullulan | silicone polymer |
| B48. | PIB | pullulan | silicone polymer mixture |
| B49. | PIB | pullulan | acrylate copolymer |
| B50. | PIB | pullulan | acrylic ester copolymer |
| B51. | PIB | pullulan | butyl rubber |
| B52. | PIB | pullulan | PIB/polybutylene |
| B53. | PIB | pullulan | styrene-iosprene |
| B54. | PIB | pullulan | styrene-butadiene |
| B55. | PIB | water-soluble | PIB |
| | | cellulose derivative | |
| B56. | PIB | water-soluble | silicone polymer |
| | | cellulose derivative | |
| B57. | PIB | water-soluble | silicone polymer mixture |
| | | cellulose derivative | |
| B58. | PIB | water-soluble | acrylate copolymer |
| | | cellulose derivative | |
| B59. | PIB | water-soluble | acrylic ester copolymer |
| | | cellulose derivative | |
| B60. | PIB | water-soluble | butyl rubber |
| | | cellulose derivative | |
| B61. | PIB | water-soluble | PIB/polybutylene |
| | | cellulose derivative | |
| B62. | PIB | water-soluble | styrene-iosprene |
| | | cellulose derivative | |
| B63. | PIB | water-soluble | styrene-butadiene |
| | | cellulose derivative | |
| B64. | PIB | polyvinyl acetate, | PIB |
| | | p.h. | |
| B65. | PIB | polyvinyl acetate, | silicone polymer |
| | | p.h. | |
| B66. | PIB | polyvinyl acetate, | silicone polymer mixture |
| | | p.h. | |
| B67. | PIB | polyvinyl acetate, | acrylate copolymer |
| | | p.h. | |
| B68. | PIB | polyvinyl acetate, | acrylic ester copolymer |
| | | p.h. | |
| B69. | PIB | polyvinyl acetate, | butyl rubber |
| | | p.h. | |
| B70. | PIB | polyvinyl acetate, | PIB/polybutylene |
| | | p.h. | |
| B71. | PIB | polyvinyl acetate, | styrene-iosprene |
| | | p.h. | |
| B72. | PIB | polyvinyl acetate, | styrene-butadiene |
| | | p.h. | |
| B73. | silicone polymer | poly(vinylpyrrolidone- | PIB |
| | | co-vinyl alcohol | |
| B74. | silicone polymer | poly(vinylpyrrolidone- | silicone polymer |
| | | co-vinyl alcohol | |
| B75. | silicone polymer | poly(vinylpyrrolidone- | silicone polymer mixture |
| | | co-vinyl alcohol | |
| B76. | silicone polymer | poly(vinylpyrrolidone- | acrylate copolymer |
| | | co-vinyl alcohol | |
| B77. | silicone polymer | poly(vinylpyrrolidone- | acrylic ester copolymer |
| | | co-vinyl alcohol | |
| B78. | silicone polymer | poly(vinylpyrrolidone- | butyl rubber |
| | | co-vinyl alcohol | |
| B79. | silicone polymer | poly(vinylpyrrolidone- | PIB/polybutylene |
| | | co-vinyl alcohol | |
| B80. | silicone polymer | poly(vinylpyrrolidone- | styrene-iosprene |
| | | co-vinyl alcohol | |
| B81. | silicone polymer | poly(vinylpyrrolidone- | styrene-butadiene |
| | | co-vinyl alcohol | |
| B82. | silicone polymer | alginate | PIB |
| B83. | silicone polymer | alginate | silicone polymer |
| B84. | silicone polymer | alginate | silicone polymer mixture |
| B85. | silicone polymer | alginate | acrylate copolymer |
| B86. | silicone polymer | alginate | acrylic ester copolymer |
| B87. | silicone polymer | alginate | butyl rubber |
| B88. | silicone polymer | alginate | PIB/polybutylene |
| B89. | silicone polymer | alginate | styrene-iosprene |
| B90. | silicone polymer | alginate | styrene-butadiene |
| B91. | silicone polymer | PVA | PIB |
| B92. | silicone polymer | PVA | silicone polymer |
| B93. | silicone polymer | PVA | silicone polymer mixture |
| B94. | silicone polymer | PVA | acrylate copolymer |
| B95. | silicone polymer | PVA | acrylic ester copolymer |
| B96. | silicone polymer | PVA | butyl rubber |
| B97. | silicone polymer | PVA | PIB/polybutylene |
| B98. | silicone polymer | PVA | styrene-iosprene |
| B99. | silicone polymer | PVA | styrene-butadiene |
| B100. | silicone polymer | PVP | PIB |
| B101. | silicone polymer | PVP | silicone polymer |
| B102. | silicone polymer | PVP | silicone polymer mixture |
| B103. | silicone polymer | PVP | acrylate copolymer |
| B104. | silicone polymer | PVP | acrylic ester copolymer |
| B105. | silicone polymer | PVP | butyl rubber |
| B106. | silicone polymer | PVP | PIB/polybutylene |
| B107. | silicone polymer | PVP | styrene-iosprene |
| B108. | silicone polymer | PVP | styrene-butadiene |
| B109. | silicone polymer | water-soluble | PIB |
| | | starch derivative | |
| B110. | silicone polymer | water-soluble | silicone polymer |
| | | starch derivative | |
| B111. | silicone polymer | water-soluble | silicone polymer mixture |
| | | starch derivative | |
| B112. | silicone polymer | water-soluble | acrylate copolymer |
| | | starch derivative | |
| B113. | silicone polymer | water-soluble | acrylic ester copolymer |
| | | starch derivative | |
| B114. | silicone polymer | water-soluble | butyl rubber |
| | | starch derivative | |
| B115. | silicone polymer | water-soluble | PIB/polybutylene |
| | | starch derivative | |
| B116. | silicone polymer | water-soluble | styrene-iosprene |
| | | starch derivative | |
| B117. | silicone polymer | water-soluble | styrene-butadiene |
| | | starch derivative | |
| B118. | silicone polymer | pullulan | PIB |
| B119. | silicone polymer | pullulan | silicone polymer |
| B120. | silicone polymer | pullulan | silicone polymer mixture |
| B121. | silicone polymer | pullulan | acrylate copolymer |
| B122. | silicone polymer | pullulan | acrylic ester copolymer |
| B123. | silicone polymer | pullulan | butyl rubber |
| B124. | silicone polymer | pullulan | PIB/polybutylene |
| B125. | silicone polymer | pullulan | styrene-iosprene |
| B126. | silicone polymer | pullulan | styrene-butadiene |
| B127. | silicone polymer | water-soluble | PIB |
| | | cellulose derivative | |
| B128. | silicone polymer | water-soluble | silicone polymer |
| | | cellulose derivative | |
| B129. | silicone polymer | water-soluble | silicone polymer mixture |
| | | cellulose derivative | |
| B130. | silicone polymer | water-soluble | acrylate copolymer |
| | | cellulose derivative | |
| B131. | silicone polymer | water-soluble | acrylic ester copolymer |
| | | cellulose derivative | |
| B132. | silicone polymer | water-soluble | butyl rubber |
| | | cellulose derivative | |
| B133. | silicone polymer | water-soluble | PIB/polybutylene |
| | | cellulose derivative | |
| B134. | silicone polymer | water-soluble | styrene-iosprene |
| | | cellulose derivative | |
| B135. | silicone polymer | water-soluble | styrene-butadiene |
| | | cellulose derivative | |
| B136. | silicone polymer | polyvinyl acetate, | PIB |
| | | p.h. | |
| B137. | silicone polymer | polyvinyl acetate, | silicone polymer |
| | | p.h. | |
| B138. | silicone polymer | polyvinyl acetate, | silicone polymer mixture |
| | | p.h. | |
| B139. | silicone polymer | polyvinyl acetate, | acrylate copolymer |
| | | p.h. | |
| B140. | silicone polymer | polyvinyl acetate, | acrylic ester copolymer |
| | | p.h. | |
| B141. | silicone polymer | polyvinyl acetate, | butyl rubber |
| | | p.h. | |
| B142. | silicone polymer | polyvinyl acetate, | PIB/polybutylene |
| | | p.h. | |
| B143. | silicone polymer | polyvinyl acetate, | styrene-iosprene |
| | | p.h. | |
| B144. | silicone polymer | polyvinyl acetate, | styrene-butadiene |
| | | p.h. | |
| B145. | silicone polymer | poly(vinylpyrrolidone- | PIB |
| | mixture | co-vinyl alcohol | |
| B146. | silicone polymer | poly(vinylpyrrolidone- | silicone polymer |
| | mixture | co-vinyl alcohol | |
| B147. | silicone polymer | poly(vinylpyrrolidone- | silicone polymer mixture |
| | mixture | co-vinyl alcohol | |
| B148. | silicone polymer | poly(vinylpyrrolidone- | acrylate copolymer |
| | mixture | co-vinyl alcohol | |
| B149. | silicone polymer | poly(vinylpyrrolidone- | acrylic ester copolymer |
| | mixture | co-vinyl alcohol | |
| B150. | silicone polymer | poly(vinylpyrrolidone- | butyl rubber |
| | mixture | co-vinyl alcohol | |
| B151. | silicone polymer | poly(vinylpyrrolidone- | PIB/polybutylene |
| | mixture | co-vinyl alcohol | |
| B152. | silicone polymer | poly(vinylpyrrolidone- | styrene-iosprene |
| | mixture | co-vinyl alcohol | |
| B153. | silicone polymer | poly(vinylpyrrolidone- | styrene-butadiene |
| | mixture | co-vinyl alcohol | |
| B154. | silicone polymer | alginate | PIB |
| | mixture | | |
| B155. | silicone polymer | alginate | silicone polymer |
| | mixture | | |
| B156. | silicone polymer | alginate | silicone polymer mixture |
| | mixture | | |
| B157. | silicone polymer | alginate | acrylate copolymer |
| | mixture | | |
| B158. | silicone polymer | alginate | acrylic ester copolymer |
| | mixture | | |
| B159. | silicone polymer | alginate | butyl rubber |
| | mixture | | |
| B160. | silicone polymer | alginate | PIB/polybutylene |
| | mixture | | |
| B161. | silicone polymer | alginate | styrene-iosprene |
| | mixture | | |
| B162. | silicone polymer | alginate | styrene-butadiene |
| | mixture | | |
| B163. | silicone polymer | PVA | PIB |
| | mixture | | |
| B164. | silicone polymer | PVA | silicone polymer |
| | mixture | | |
| B165. | silicone polymer | PVA | silicone polymer mixture |
| | mixture | | |
| B166. | silicone polymer | PVA | acrylate copolymer |
| | mixture | | |
| B167. | silicone polymer | PVA | acrylic ester copolymer |
| | mixture | | |
| B168. | silicone polymer | PVA | butyl rubber |
| | mixture | | |
| B169. | silicone polymer | PVA | PIB/polybutylene |
| | mixture | | |
| B170. | silicone polymer | PVA | styrene-iosprene |
| | mixture | | |
| B171. | silicone polymer | PVA | styrene-butadiene |
| | mixture | | |
| B172. | silicone polymer | PVP | PIB |
| | mixture | | |
| B173. | silicone polymer | PVP | silicone polymer |
| | mixture | | |
| B174. | silicone polymer | PVP | silicone polymer mixture |
| | mixture | | |
| B175. | silicone polymer | PVP | acrylate copolymer |
| | mixture | | |
| B176. | silicone polymer | PVP | acrylic ester copolymer |
| | mixture | | |
| B177. | silicone polymer | PVP | butyl rubber |
| | mixture | | |
| B178. | silicone polymer | PVP | PIB/polybutylene |
| | mixture | | |
| B179. | silicone polymer | PVP | styrene-iosprene |
| | mixture | | |
| B180. | silicone polymer | PVP | styrene-butadiene |
| | mixture | | |
| B181. | silicone polymer | water-soluble | PIB |
| | mixture | starch derivative | |
| B182. | silicone polymer | water-soluble | silicone polymer |
| | mixture | starch derivative | |
| B183. | silicone polymer | water-soluble | silicone polymer mixture |
| | mixture | starch derivative | |
| B184. | silicone polymer | water-soluble | acrylate copolymer |
| | mixture | starch derivative | |
| B185. | silicone polymer | water-soluble | acrylic ester copolymer |
| | mixture | starch derivative | |
| B186. | silicone polymer | water-soluble | butyl rubber |
| | mixture | starch derivative | |
| B187. | silicone polymer | water-soluble | PIB/polybutylene |
| | mixture | starch derivative | |
| B188. | silicone polymer | water-soluble | styrene-iosprene |
| | mixture | starch derivative | |
| B189. | silicone polymer | water-soluble | styrene-butadiene |
| | mixture | starch derivative | |
| B190. | silicone polymer | pullulan | PIB |
| | mixture | | |
| B191. | silicone polymer | pullulan | silicone polymer |
| | mixture | | |
| B192. | silicone polymer | pullulan | silicone polymer mixture |
| | mixture | | |
| B193. | silicone polymer | pullulan | acrylate copolymer |
| | mixture | | |
| B194. | silicone polymer | pullulan | acrylic ester copolymer |
| | mixture | | |
| B195. | silicone polymer | pullulan | butyl rubber |
| | mixture | | |
| B196. | silicone polymer | pullulan | PIB/polybutylene |
| | mixture | | |
| B197. | silicone polymer | Pullulan | styrene-iosprene |
| | mixture | | |
| B198. | silicone polymer | pullulan | styrene-butadiene |
| | mixture | | |
| B199. | silicone polymer | water-soluble | PIB |
| | mixture | cellulose derivative | |
| B200. | silicone polymer | water-soluble | silicone polymer |
| | mixture | cellulose derivative | |
| B201. | silicone polymer | water-soluble | silicone polymer mixture |
| | mixture | cellulose derivative | |
| B202. | silicone polymer | water-soluble | acrylate copolymer |
| | mixture | cellulose derivative | |
| B203. | silicone polymer | water-soluble | acrylic ester copolymer |
| | mixture | cellulose derivative | |
| B204. | silicone polymer | water-soluble | butyl rubber |
| | mixture | cellulose derivative | |
| B205. | silicone polymer | water-soluble | PIB/polybutylene |
| | mixture | cellulose derivative | |
| B206. | silicone polymer | water-soluble | styrene-iosprene |
| | mixture | cellulose derivative | |
| B207. | silicone polymer | water-soluble | styrene-butadiene |
| | mixture | cellulose derivative | |
| B208. | silicone polymer | polyvinyl acetate, | PIB |
| | mixture | p.h. | |
| B209. | silicone polymer | polyvinyl acetate, | silicone polymer |
| | mixture | p.h. | |
| B210. | silicone polymer | polyvinyl acetate, | silicone polymer mixture |
| | mixture | p.h. | |
| B211. | silicone polymer | polyvinyl acetate, | acrylate copolymer |
| | mixture | p.h. | |
| B212. | silicone polymer | polyvinyl acetate, | acrylic ester copolymer |
| | mixture | p.h. | |
| B213. | silicone polymer | polyvinyl acetate, | butyl rubber |
| | mixture | p.h. | |
| B214. | silicone polymer | polyvinyl acetate, | PIB/polybutylene |
| | mixture | p.h. | |
| B215. | silicone polymer | polyvinyl acetate, | styrene-iosprene |
| | mixture | p.h. | |
| B216. | silicone polymer | polyvinyl acetate, | styrene-butadiene |
| | mixture | p.h. | |
| B217. | acrylate copolymer | poly(vinylpyrrolidone- | PIB |
| | | co-vinyl alcohol | |
| B218. | acrylate copolymer | poly(vinylpyrrolidone- | silicone polymer |
| | | co-vinyl alcohol | |
| B219. | acrylate copolymer | poly(vinylpyrrolidone- | silicone polymer mixture |
| | | co-vinyl alcohol | |
| B220. | acrylate copolymer | poly(vinylpyrrolidone- | acrylate copolymer |
| | | co-vinyl alcohol | |
| B221. | acrylate copolymer | poly(vinylpyrrolidone- | acrylic ester copolymer |
| | | co-vinyl alcohol | |
| B222. | acrylate copolymer | poly(vinylpyrrolidone- | butyl rubber |
| | | co-vinyl alcohol | |
| B223. | acrylate copolymer | poly(vinylpyrrolidone- | PIB/polybutylene |
| | | co-vinyl alcohol | |
| B224. | acrylate copolymer | poly(vinylpyrrolidone- | styrene-iosprene |
| | | co-vinyl alcohol | |
| B225. | acrylate copolymer | poly(vinylpyrrolidone- | styrene-butadiene |
| | | co-vinyl alcohol | |
| B226. | acrylate copolymer | alginate | PIB |
| B227. | acrylate copolymer | alginate | silicone polymer |
| B228. | acrylate copolymer | alginate | silicone polymer mixture |
| B229. | acrylate copolymer | alginate | acrylate copolymer |
| B230. | acrylate copolymer | alginate | acrylic ester copolymer |
| B231. | acrylate copolymer | alginate | butyl rubber |
| B232. | acrylate copolymer | alginate | PIB/polybutylene |
| B233. | acrylate copolymer | alginate | styrene-iosprene |
| B234. | acrylate copolymer | alginate | styrene-butadiene |
| B235. | acrylate copolymer | PVA | PIB |
| B236. | acrylate copolymer | PVA | silicone polymer |
| B237. | acrylate copolymer | PVA | silicone polymer mixture |
| B238. | acrylate copolymer | PVA | acrylate copolymer |
| B239. | acrylate copolymer | PVA | acrylic ester copolymer |
| B240. | acrylate copolymer | PVA | butyl rubber |
| B241. | acrylate copolymer | PVA | PIB/polybutylene |
| B242. | acrylate copolymer | PVA | styrene-iosprene |
| B243. | acrylate copolymer | PVA | styrene-butadiene |
| B244. | acrylate copolymer | PVP | PIB |
| B245. | acrylate copolymer | PVP | silicone polymer |
| B246. | acrylate copolymer | PVP | silicone polymer mixture |
| B247. | acrylate copolymer | PVP | acrylate copolymer |
| B248. | acrylate copolymer | PVP | acrylic ester copolymer |
| B249. | acrylate copolymer | PVP | butyl rubber |
| B250. | acrylate copolymer | PVP | PIB/polybutylene |
| B251. | acrylate copolymer | PVP | styrene-iosprene |
| B252. | acrylate copolymer | PVP | styrene-butadiene |
| B253. | acrylate copolymer | water-soluble | PIB |
| | | starch derivative | |
| B254. | acrylate copolymer | water-soluble | silicone polymer |
| | | starch derivative | |
| B255. | acrylate copolymer | water-soluble | silicone polymer mixture |
| | | starch derivative | |
| B256. | acrylate copolymer | water-soluble | acrylate copolymer |
| | | starch derivative | |
| B257. | acrylate copolymer | water-soluble | acrylic ester copolymer |
| | | starch derivative | |
| B258. | acrylate copolymer | water-soluble | butyl rubber |
| | | starch derivative | |
| B259. | acrylate copolymer | water-soluble | PIB/polybutylene |
| | | starch derivative | |
| B260. | acrylate copolymer | water-soluble | styrene-iosprene |
| | | starch derivative | |
| B261. | acrylate copolymer | water-soluble | styrene-butadiene |
| | | starch derivative | |
| B262. | acrylate copolymer | pullulan | PIB |
| B263. | acrylate copolymer | pullulan | silicone polymer |
| B264. | acrylate copolymer | pullulan | silicone polymer mixture |
| B265. | acrylate copolymer | pullulan | acrylate copolymer |
| B266. | acrylate copolymer | pullulan | acrylic ester copolymer |
| B267. | acrylate copolymer | pullulan | butyl rubber |
| B268. | acrylate copolymer | pullulan | PIB/polybutylene |
| B269. | acrylate copolymer | pullulan | styrene-iosprene |
| B270. | acrylate copolymer | pullulan | styrene-butadiene |
| B271. | acrylate copolymer | water-soluble | PIB |
| | | cellulose derivative | |
| B272. | acrylate copolymer | water-soluble | silicone polymer |
| | | cellulose derivative | |
| B273. | acrylate copolymer | water-soluble | silicone polymer mixture |
| | | cellulose derivative | |
| B274. | acrylate copolymer | water-soluble | acrylate copolymer |
| | | cellulose derivative | |
| B275. | acrylate copolymer | water-soluble | acrylic ester copolymer |
| | | cellulose derivative | |
| B276. | acrylate copolymer | water-soluble | butyl rubber |
| | | cellulose derivative | |
| B277. | acrylate copolymer | water-soluble | PIB/polybutylene |
| | | cellulose derivative | |
| B278. | acrylate copolymer | water-soluble | styrene-iosprene |
| | | cellulose derivative | |
| B279. | acrylate copolymer | water-soluble | styrene-butadiene |
| | | cellulose derivative | |
| B280. | acrylate copolymer | polyvinyl acetate, | PIB |
| | | p.h. | |
| B281. | acrylate copolymer | polyvinyl acetate, | silicone polymer |
| | | p.h. | |
| B282. | acrylate copolymer | polyvinyl acetate, | silicone polymer mixture |
| | | p.h. | |
| B283. | acrylate copolymer | polyvinyl acetate, | acrylate copolymer |
| | | p.h. | |
| B284. | acrylate copolymer | polyvinyl acetate, | acrylic ester copolymer |
| | | p.h. | |
| B285. | acrylate copolymer | polyvinyl acetate, | butyl rubber |
| | | p.h. | |
| B286. | acrylate copolymer | polyvinyl acetate, | PIB/polybutylene |
| | | p.h. | |
| B287. | acrylate copolymer | polyvinyl acetate, | styrene-iosprene |
| | | p.h. | |
| B288. | acrylate copolymer | polyvinyl acetate, | styrene-butadiene |
| | | p.h. | |
| B289. | acrylic ester copolymer | poly(vinylpyrrolidone- | PIB |
| | | co-vinyl alcohol | |
| B290. | acrylic ester | poly(vinylpyrrolidone- | silicone polymer |
| | copolymer | co-vinyl alcohol | |
| B291. | acrylic ester | poly(vinylpyrrolidone- | silicone polymer mixture |
| | copolymer | co-vinyl alcohol | |
| B292. | acrylic ester | poly(vinylpyrrolidone- | acrylate copolymer |
| | copolymer | co-vinyl alcohol | |
| B293. | acrylic ester | poly(vinylpyrrolidone- | acrylic ester copolymer |
| | copolymer | co-vinyl alcohol | |
| B294. | acrylic ester | poly(vinylpyrrolidone- | butyl rubber |
| | copolymer | co-vinyl alcohol | |
| B295. | acrylic ester | poly(vinylpyrrolidone- | PIB/polybutylene |
| | copolymer | co-vinyl alcohol | |
| B296. | acrylic ester | poly(vinylpyrrolidone- | styrene-iosprene |
| | copolymer | co-vinyl alcohol | |
| B297. | acrylic ester | poly(vinylpyrrolidone- | styrene-butadiene |
| | copolymer | co-vinyl alcohol | |
| B298. | acrylic ester | alginate | PIB |
| | copolymer | | |
| B299. | acrylic ester | alginate | silicone polymer |
| | copolymer | | |
| B300. | acrylic ester | alginate | silicone polymer mixture |
| | copolymer | | |
| B301. | acrylic ester | alginate | acrylate copolymer |
| | copolymer | | |
| B302. | acrylic ester | alginate | acrylic ester copolymer |
| | copolymer | | |
| B303. | acrylic ester | alginate | butyl rubber |
| | copolymer | | |
| B304. | acrylic ester | alginate | PIB/polybutylene |
| | copolymer | | |
| B305. | acrylic ester | alginate | styrene-iosprene |
| | copolymer | | |
| B306. | acrylic ester | alginate | styrene-butadiene |
| | copolymer | | |
| B307. | acrylic ester | PVA | PIB |
| | copolymer | | |
| B308. | acrylic ester | PVA | silicone polymer |
| | copolymer | | |
| B309. | acrylic ester | PVA | silicone polymer mixture |
| | copolymer | | |
| B310. | acrylic ester | PVA | acrylate copolymer |
| | copolymer | | |
| B311. | acrylic ester | PVA | acrylic ester copolymer |
| | copolymer | | |
| B312. | acrylic ester | PVA | butyl rubber |
| | copolymer | | |
| B313. | acrylic ester | PVA | PIB/polybutylene |
| | copolymer | | |
| B314. | acrylic ester | PVA | styrene-iosprene |
| | copolymer | | |
| B315. | acrylic ester | PVA | styrene-butadiene |
| | copolymer | | |
| B316. | acrylic ester | PVP | PIB |
| | copolymer | | |
| B317. | acrylic ester | PVP | silicone polymer |
| | copolymer | | |
| B318. | acrylic ester | PVP | silicone polymer mixture |
| | copolymer | | |
| B319. | acrylic ester | PVP | acrylate copolymer |
| | copolymer | | |
| B320. | acrylic ester | PVP | acrylic ester copolymer |
| | copolymer | | |
| B321. | acrylic ester | PVP | butyl rubber |
| | copolymer | | |
| B322. | acrylic ester | PVP | PIB/polybutylene |
| | copolymer | | |
| B323. | acrylic ester | PVP | styrene-iosprene |
| | copolymer | | |
| B324. | acrylic ester | PVP | styrene-butadiene |
| | copolymer | | |
| B325. | acrylic ester | water-soluble | PIB |
| | copolymer | starch derivative | |
| B326. | acrylic ester | water-soluble | silicone polymer |
| | copolymer | starch derivative | |
| B327. | acrylic ester | water-soluble | silicone polymer mixture |
| | copolymer | starch derivative | |
| B328. | acrylic ester | water-soluble | acrylate copolymer |
| | copolymer | starch derivative | |
| B329. | acrylic ester | water-soluble | acrylic ester copolymer |
| | copolymer | starch derivative | |
| B330. | acrylic ester | water-soluble | butyl rubber |
| | copolymer | starch derivative | |
| B331. | acrylic ester | water-soluble | PIB/polybutylene |
| | copolymer | starch derivative | |
| B332. | acrylic ester | water-soluble | styrene-iosprene |
| | copolymer | starch derivative | |
| B333. | acrylic ester | water-soluble | styrene-butadiene |
| | copolymer | starch derivative | |
| B334. | acrylic ester | pullulan | PIB |
| | copolymer | | |
| B335. | acrylic ester | pullulan | silicone polymer |
| | copolymer | | |
| B336. | acrylic ester | pullulan | silicone polymer mixture |
| | copolymer | | |
| B337. | acrylic ester | pullulan | acrylate copolymer |
| | copolymer | | |
| B338. | acrylic ester | pullulan | acrylic ester copolymer |
| | copolymer | | |
| B339. | acrylic ester | pullulan | butyl rubber |
| | copolymer | | |
| B340. | acrylic ester | pullulan | PIB/polybutylene |
| | copolymer | | |
| B341. | acrylic ester | pullulan | styrene-iosprene |
| | copolymer | | |
| B342. | acrylic ester | pullulan | styrene-butadiene |
| | copolymer | | |
| B343. | acrylic ester | water-soluble | PIB |
| | copolymer | cellulose derivative | |
| B344. | acrylic ester | water-soluble | silicone polymer |
| | copolymer | cellulose derivative | |
| B345. | acrylic ester | water-soluble | silicone polymer mixture |
| | copolymer | cellulose derivative | |
| B346. | acrylic ester | water-soluble | acrylate copolymer |
| | copolymer | cellulose derivative | |
| B347. | acrylic ester | water-soluble | acrylic ester copolymer |
| | copolymer | cellulose derivative | |
| B348. | acrylic ester | water-soluble | butyl rubber |
| | copolymer | cellulose derivative | |
| B349. | acrylic ester | water-soluble | PIB/polybutylene |
| | copolymer | cellulose derivative | |
| B350. | acrylic ester | water-soluble | styrene-iosprene |
| | copolymer | cellulose derivative | |
| B351. | acrylic ester | water-soluble | styrene-butadiene |
| | copolymer | cellulose derivative | |
| B352. | acrylic ester | polyvinyl acetate, | PIB |
| | copolymer | p.h. | |
| B353. | acrylic ester | polyvinyl acetate, | silicone polymer |
| | copolymer | p.h. | |
| B354. | acrylic ester | polyvinyl acetate, | silicone polymer mixture |
| | copolymer | p.h. | |
| B355. | acrylic ester | polyvinyl acetate, | acrylate copolymer |
| | copolymer | p.h. | |
| B356. | acrylic ester | polyvinyl acetate, | acrylic ester copolymer |
| | copolymer | p.h. | |
| B357. | acrylic ester | polyvinyl acetate, | butyl rubber |
| | copolymer | p.h. | |
| B358. | acrylic ester | polyvinyl acetate, | PIB/polybutylene |
| | copolymer | p.h. | |
| B359. | acrylic ester | polyvinyl acetate, | styrene-iosprene |
| | copolymer | p.h. | |
| B360. | acrylic ester | polyvinyl acetate, | styrene-butadiene |
| | copolymer | p.h. | |
| B361. | butyl rubber | poly(vinylpyrrolidone- | PIB |
| | | co-vinyl alcohol | |
| B362. | butyl rubber | poly(vinylpyrrolidone- | silicone polymer |
| | | co-vinyl alcohol | |
| B363. | butyl rubber | poly(vinylpyrrolidone- | silicone polymer mixture |
| | | co-vinyl alcohol | |
| B364. | butyl rubber | poly(vinylpyrrolidone- | acrylate copolymer |
| | | co-vinyl alcohol | |
| B365. | butyl rubber | poly(vinylpyrrolidone- | acrylic ester copolymer |
| | | co-vinyl alcohol | |
| B366. | butyl rubber | poly(vinylpyrrolidone- | butyl rubber |
| | | co-vinyl alcohol | |
| B367. | butyl rubber | poly(vinylpyrrolidone- | PIB/polybutylene |
| | | co-vinyl alcohol | |
| B368. | butyl rubber | poly(vinylpyrrolidone- | styrene-iosprene |
| | | co-vinyl alcohol | |
| B369. | butyl rubber | poly(vinylpyrrolidone- | styrene-butadiene |
| | | co-vinyl alcohol | |
| B370. | butyl rubber | alginate | PIB |
| B371. | butyl rubber | alginate | silicone polymer |
| B372. | butyl rubber | alginate | silicone polymer mixture |
| B373. | butyl rubber | alginate | acrylate copolymer |
| B374. | butyl rubber | alginate | acrylic ester copolymer |
| B375. | butyl rubber | alginate | butyl rubber |
| B376. | butyl rubber | alginate | PIB/polybutylene |
| B377. | butyl rubber | alginate | styrene-iosprene |
| B378. | butyl rubber | alginate | styrene-butadiene |
| B379. | butyl rubber | PVA | PIB |
| B380. | butyl rubber | PVA | silicone polymer |
| B381. | butyl rubber | PVA | silicone polymer mixture |
| B382. | butyl rubber | PVA | acrylate copolymer |
| B383. | butyl rubber | PVA | acrylic ester copolymer |
| B384. | butyl rubber | PVA | butyl rubber |
| B385. | butyl rubber | PVA | PIB/polybutylene |
| B386. | butyl rubber | PVA | styrene-iosprene |
| B387. | butyl rubber | PVA | styrene-butadiene |
| B388. | butyl rubber | PVP | PIB |
| B389. | butyl rubber | PVP | silicone polymer |
| B390. | butyl rubber | PVP | silicone polymer mixture |
| B391. | butyl rubber | PVP | acrylate copolymer |
| B392. | butyl rubber | PVP | acrylic ester copolymer |
| B393. | butyl rubber | PVP | butyl rubber |
| B394. | butyl rubber | PVP | PIB/polybutylene |
| B395. | butyl rubber | PVP | styrene-iosprene |
| B396. | butyl rubber | PVP | styrene-butadiene |
| B397. | butyl rubber | water-soluble | PIB |
| | | starch derivative | |
| B398. | butyl rubber | water-soluble | silicone polymer |
| | | starch derivative | |
| B399. | butyl rubber | water-soluble | silicone polymer mixture |
| | | starch derivative | |
| B400. | butyl rubber | water-soluble | acrylate copolymer |
| | | starch derivative | |
| B401. | butyl rubber | water-soluble | acrylic ester copolymer |
| | | starch derivative | |
| B402. | butyl rubber | water-soluble | butyl rubber |
| | | starch derivative | |
| B403. | butyl rubber | water-soluble | PIB/polybutylene |
| | | starch derivative | |
| B404. | butyl rubber | water-soluble | styrene-iosprene |
| | | starch derivative | |
| B405. | butyl rubber | water-soluble | styrene-butadiene |
| | | starch derivative | |
| B406. | butyl rubber | pullulan | PIB |
| B407. | butyl rubber | pullulan | silicone polymer |
| B408. | butyl rubber | pullulan | silicone polymer mixture |
| B409. | butyl rubber | pullulan | acrylate copolymer |
| B410. | butyl rubber | pullulan | acrylic ester copolymer |
| B411. | butyl rubber | pullulan | butyl rubber |
| B412. | butyl rubber | pullulan | PIB/polybutylene |
| B413. | butyl rubber | pullulan | styrene-iosprene |
| B414. | butyl rubber | pullulan | styrene-butadiene |
| B415. | butyl rubber | water-soluble | PIB |
| | | cellulose derivative | |
| B416. | butyl rubber | water-soluble | silicone polymer |
| | | cellulose derivative | |
| B417. | butyl rubber | water-soluble | silicone polymer mixture |
| | | cellulose derivative | |
| B418. | butyl rubber | water-soluble | acrylate copolymer |
| | | cellulose derivative | |
| B419. | butyl rubber | water-soluble | acrylic ester copolymer |
| | | cellulose derivative | |
| B420. | butyl rubber | water-soluble | butyl rubber |
| | | cellulose derivative | |
| B421. | butyl rubber | water-soluble | PIB/polybutylene |
| | | cellulose derivative | |
| B422. | butyl rubber | water-soluble | styrene-iosprene |
| | | cellulose derivative | |
| B423. | butyl rubber | water-soluble | styrene-butadiene |
| | | cellulose derivative | |
| B424. | butyl rubber | polyvinyl acetate, | PIB |
| | | p.h. | |
| B425. | butyl rubber | polyvinyl acetate, | silicone polymer |
| | | p.h. | |
| B426. | butyl rubber | polyvinyl acetate, | silicone polymer mixture |
| | | p.h. | |
| B427. | butyl rubber | polyvinyl acetate, | acrylate copolymer |
| | | p.h. | |
| B428. | butyl rubber | polyvinyl acetate, | acrylic ester copolymer |
| | | p.h. | |
| B429. | butyl rubber | polyvinyl acetate, | butyl rubber |
| | | p.h. | |
| B430. | butyl rubber | polyvinyl acetate, | PIB/polybutylene |
| | | p.h. | |
| B431. | butyl rubber | polyvinyl acetate, | styrene-iosprene |
| | | p.h. | |
| B432. | butyl rubber | polyvinyl acetate, | styrene-butadiene |
| | | p.h. | |
| B433. | PIB/polybutylene | poly(vinylpyrrolidone- | PIB |
| | | co-vinyl alcohol | |
| B434. | PIB/polybutylene | poly(vinylpyrrolidone- | silicone polymer |
| | | co-vinyl alcohol | |
| B435. | PIB/polybutylene | poly(vinylpyrrolidone- | silicone polymer mixture |
| | | co-vinyl alcohol | |
| B436. | PIB/polybutylene | poly(vinylpyrrolidone- | acrylate copolymer |
| | | co-vinyl alcohol | |
| B437. | PIB/polybutylene | poly(vinylpyrrolidone- | acrylic ester copolymer |
| | | co-vinyl alcohol | |
| B438. | PIB/polybutylene | poly(vinylpyrrolidone- | butyl rubber |
| | | co-vinyl alcohol | |
| B439. | PIB/polybutylene | poly(vinylpyrrolidone- | PIB/polybutylene |
| | | co-vinyl alcohol | |
| B440. | PIB/polybutylene | poly(vinylpyrrolidone- | styrene-iosprene |
| | | co-vinyl alcohol | |
| B441. | PIB/polybutylene | poly(vinylpyrrolidone- | styrene-butadiene |
| | | co-vinyl alcohol | |
| B442. | PIB/polybutylene | alginate | PIB |
| B443. | PIB/polybutylene | alginate | silicone polymer |
| B444. | PIB/polybutylene | alginate | silicone polymer mixture |
| B445. | PIB/polybutylene | alginate | acrylate copolymer |
| B446. | PIB/polybutylene | alginate | acrylic ester copolymer |
| B447. | PIB/polybutylene | alginate | butyl rubber |
| B448. | PIB/polybutylene | alginate | PIB/polybutylene |
| B449. | PIB/polybutylene | alginate | styrene-iosprene |
| B450. | PIB/polybutylene | alginate | styrene-butadiene |
| B451. | PIB/polybutylene | PVA | PIB |
| B452. | PIB/polybutylene | PVA | silicone polymer |
| B453. | PIB/polybutylene | PVA | silicone polymer mixture |
| B454. | PIB/polybutylene | PVA | acrylate copolymer |
| B455. | PIB/polybutylene | PVA | acrylic ester copolymer |
| B456. | PIB/polybutylene | PVA | butyl rubber |
| B457. | PIB/polybutylene | PVA | PIB/polybutylene |
| B458. | PIB/polybutylene | PVA | styrene-iosprene |
| B459. | PIB/polybutylene | PVA | styrene-butadiene |
| B460. | PIB/polybutylene | PVP | PIB |
| B461. | PIB/polybutylene | PVP | silicone polymer |
| B462. | PIB/polybutylene | PVP | silicone polymer mixture |
| B463. | PIB/polybutylene | PVP | acrylate copolymer |
| B464. | PIB/polybutylene | PVP | acrylic ester copolymer |
| B465. | PIB/polybutylene | PVP | butyl rubber |
| B466. | PIB/polybutylene | PVP | PIB/polybutylene |
| B467. | PIB/polybutylene | PVP | styrene-iosprene |
| B468. | PIB/polybutylene | PVP | styrene-butadiene |
| B469. | PIB/polybutylene | water-soluble | PIB |
| | | starch derivative | |
| B470. | PIB/polybutylene | water-soluble | silicone polymer |
| | | starch derivative | |
| B471. | PIB/polybutylene | water-soluble | silicone polymer mixture |
| | | starch derivative | |
| B472. | PIB/polybutylene | water-soluble | acrylate copolymer |
| | | starch derivative | |
| B473. | PIB/polybutylene | water-soluble | acrylic ester copolymer |
| | | starch derivative | |
| B474. | PIB/polybutylene | water-soluble | butyl rubber |
| | | starch derivative | |
| B475. | PIB/polybutylene | water-soluble | PIB/polybutylene |
| | | starch derivative | |
| B476. | PIB/polybutylene | water-soluble | styrene-iosprene |
| | | starch derivative | |
| B477. | PIB/polybutylene | water-soluble | styrene-butadiene |
| | | starch derivative | |
| B478. | PIB/polybutylene | pullulan | PIB |
| B479. | PIB/polybutylene | pullulan | silicone polymer |
| B480. | PIB/polybutylene | pullulan | silicone polymer mixture |
| B481. | PIB/polybutylene | pullulan | acrylate copolymer |
| B482. | PIB/polybutylene | pullulan | acrylic ester copolymer |
| B483. | PIB/polybutylene | pullulan | butyl rubber |
| B484. | PIB/polybutylene | pullulan | PIB/polybutylene |
| B485. | PIB/polybutylene | pullulan | styrene-iosprene |
| B486. | PIB/polybutylene | pullulan | styrene-butadiene |
| B487. | PIB/polybutylene | water-soluble | PIB |
| | | cellulose derivative | |
| B488. | PIB/polybutylene | water-soluble | silicone polymer |
| | | cellulose derivative | |
| B489. | PIB/polybutylene | water-soluble | silicone polymer mixture |
| | | cellulose derivative | |
| B490. | PIB/polybutylene | water-soluble | acrylate copolymer |
| | | cellulose derivative | |
| B491. | PIB/polybutylene | water-soluble | acrylic ester copolymer |
| | | cellulose derivative | |
| B492. | PIB/polybutylene | water-soluble | butyl rubber |
| | | cellulose derivative | |
| B493. | PIB/polybutylene | water-soluble | PIB/polybutylene |
| | | cellulose derivative | |
| B494. | PIB/polybutylene | water-soluble | styrene-iosprene |
| | | cellulose derivative | |
| B495. | PIB/polybutylene | water-soluble | styrene-butadiene |
| | | cellulose derivative | |
| B496. | PIB/polybutylene | polyvinyl acetate, | PIB |
| | | p.h. | |
| B497. | PIB/polybutylene | polyvinyl acetate, | silicone polymer |
| | | p.h. | |
| B498. | PIB/polybutylene | polyvinyl acetate, | silicone polymer mixture |
| | | p.h. | |
| B499. | PIB/polybutylene | polyvinyl acetate, | acrylate copolymer |
| | | p.h. | |
| B500. | PIB/polybutylene | polyvinyl acetate, | acrylic ester copolymer |
| | | p.h. | |
| B501. | PIB/polybutylene | polyvinyl acetate, | butyl rubber |
| | | p.h. | |
| B502. | PIB/polybutylene | polyvinyl acetate, | PIB/polybutylene |
| | | p.h. | |
| B503. | PIB/polybutylene | polyvinyl acetate, | styrene-iosprene |
| | | p.h. | |
| B504. | PIB/polybutylene | polyvinyl acetate, | styrene-butadiene |
| | | p.h. | |
| B505. | styrene-iosprene | poly(vinylpyrrolidone- | PIB |
| | | co-vinyl alcohol | |
| B506. | styrene-iosprene | poly(vinylpyrrolidone- | silicone polymer |
| | | co-vinyl alcohol | |
| B507. | styrene-iosprene | poly(vinylpyrrolidone- | silicone polymer mixture |
| | | co-vinyl alcohol | |
| B508. | styrene-iosprene | poly(vinylpyrrolidone- | acrylate copolymer |
| | | co-vinyl alcohol | |
| B509. | styrene-iosprene | poly(vinylpyrrolidone- | acrylic ester copolymer |
| | | co-vinyl alcohol | |
| B510. | styrene-iosprene | poly(vinylpyrrolidone- | butyl rubber |
| | | co-vinyl alcohol | |
| B511. | styrene-iosprene | poly(vinylpyrrolidone- | PIB/polybutylene |
| | | co-vinyl alcohol | |
| B512. | styrene-iosprene | poly(vinylpyrrolidone- | styrene-iosprene |
| | | co-vinyl alcohol | |
| B513. | styrene-iosprene | poly(vinylpyrrolidone- | styrene-butadiene |
| | | co-vinyl alcohol | |
| B514. | styrene-iosprene | alginate | PIB |
| B515. | styrene-iosprene | alginate | silicone polymer |
| B516. | styrene-iosprene | alginate | silicone polymer mixture |
| B517. | styrene-iosprene | alginate | acrylate copolymer |
| B518. | styrene-iosprene | alginate | acrylic ester copolymer |
| B519. | styrene-iosprene | alginate | butyl rubber |
| B520. | styrene-iosprene | alginate | PIB/polybutylene |
| B521. | styrene-iosprene | alginate | styrene-iosprene |
| B522. | styrene-iosprene | alginate | styrene-butadiene |
| B523. | styrene-iosprene | PVA | PIB |
| B524. | styrene-iosprene | PVA | silicone polymer |
| B525. | styrene-iosprene | PVA | silicone polymer mixture |
| B526. | styrene-iosprene | PVA | acrylate copolymer |
| B527. | styrene-iosprene | PVA | acrylic ester copolymer |
| B528. | styrene-iosprene | PVA | butyl rubber |
| B529. | styrene-iosprene | PVA | PIB/polybutylene |
| B530. | styrene-iosprene | PVA | styrene-iosprene |
| B531. | styrene-iosprene | PVA | styrene-butadiene |
| B532. | styrene-iosprene | PVP | PIB |
| B533. | styrene-iosprene | PVP | silicone polymer |
| B534. | styrene-iosprene | PVP | silicone polymer mixture |
| B535. | styrene-iosprene | PVP | acrylate copolymer |
| B536. | styrene-iosprene | PVP | acrylic ester copolymer |
| B537. | styrene-iosprene | PVP | butyl rubber |
| B538. | styrene-iosprene | PVP | PIB/polybutylene |
| B539. | styrene-iosprene | PVP | styrene-iosprene |
| B540. | styrene-iosprene | PVP | styrene-butadiene |
| B541. | styrene-iosprene | water-soluble | PIB |
| | | starch derivative | |
| B542. | styrene-iosprene | water-soluble | silicone polymer |
| | | starch derivative | |
| B543. | styrene-iosprene | water-soluble | silicone polymer mixture |
| | | starch derivative | |
| B544. | styrene-iosprene | water-soluble | acrylate copolymer |
| | | starch derivative | |
| B545. | styrene-iosprene | water-soluble | acrylic ester copolymer |
| | | starch derivative | |
| B546. | styrene-iosprene | water-soluble | butyl rubber |
| | | starch derivative | |
| B547. | styrene-iosprene | water-soluble | PIB/polybutylene |
| | | starch derivative | |
| B548. | styrene-iosprene | water-soluble | styrene-iosprene |
| | | starch derivative | |
| B549. | styrene-iosprene | water-soluble | styrene-butadiene |
| | | starch derivative | |
| B550. | styrene-iosprene | pullulan | PIB |
| B551. | styrene-iosprene | pullulan | silicone polymer |
| B552. | styrene-iosprene | pullulan | silicone polymer mixture |
| B553. | styrene-iosprene | pullulan | acrylate copolymer |
| B554. | styrene-iosprene | pullulan | acrylic ester copolymer |
| B555. | styrene-iosprene | pullulan | butyl rubber |
| B556. | styrene-iosprene | pullulan | PIB/polybutylene |
| B557. | styrene-iosprene | pullulan | styrene-iosprene |
| B558. | styrene-iosprene | pullulan | styrene-butadiene |
| B559. | styrene-iosprene | water-soluble | PIB |
| | | cellulose derivative | |
| B560. | styrene-iosprene | water-soluble | silicone polymer |
| | | cellulose derivative | |
| B561. | styrene-iosprene | water-soluble | silicone polymer mixture |
| | | cellulose derivative | |
| B562. | styrene-iosprene | water-soluble | acrylate copolymer |
| | | cellulose derivative | |
| B563. | styrene-iosprene | water-soluble | acrylic ester copolymer |
| | | cellulose derivative | |
| B564. | styrene-iosprene | water-soluble | butyl rubber |
| | | cellulose derivative | |
| B565. | styrene-iosprene | water-soluble | PIB/polybutylene |
| | | cellulose derivative | |
| B566. | styrene-iosprene | water-soluble | styrene-iosprene |
| | | cellulose derivative | |
| B567. | styrene-iosprene | water-soluble | styrene-butadiene |
| | | cellulose derivative | |
| B568. | styrene-iosprene | polyvinyl acetate, | PIB |
| | | p.h. | |
| B569. | styrene-iosprene | polyvinyl acetate, | silicone polymer |
| | | p.h. | |
| B570. | styrene-iosprene | polyvinyl acetate, | silicone polymer mixture |
| | | p.h. | |
| B571. | styrene-iosprene | polyvinyl acetate, | acrylate copolymer |
| | | p.h. | |
| B572. | styrene-iosprene | polyvinyl acetate, | acrylic ester copolymer |
| | | p.h. | |
| B573. | styrene-iosprene | polyvinyl acetate, | butyl rubber |
| | | p.h. | |
| B574. | styrene-iosprene | polyvinyl acetate, | PIB/polybutylene |
| | | p.h. | |
| B575. | styrene-iosprene | polyvinyl acetate, | styrene-iosprene |
| | | p.h. | |
| B576. | styrene-iosprene | polyvinyl acetate, | styrene-butadiene |
| | | p.h. | |
| B577. | styrene-butadiene | poly(vinylpyrrolidone- | PIB |
| | | co-vinyl alcohol | |
| B578. | styrene-butadiene | poly(vinylpyrrolidone- | silicone polymer |
| | | co-vinyl alcohol | |
| B579. | styrene-butadiene | poly(vinylpyrrolidone- | silicone polymer mixture |
| | | co-vinyl alcohol | |
| B580. | styrene-butadiene | poly(vinylpyrrolidone- | acrylate copolymer |
| | | co-vinyl alcohol | |
| B581. | styrene-butadiene | poly(vinylpyrrolidone- | acrylic ester copolymer |
| | | co-vinyl alcohol | |
| B582. | styrene-butadiene | poly(vinylpyrrolidone- | butyl rubber |
| | | co-vinyl alcohol | |
| B583. | styrene-butadiene | poly(vinylpyrrolidone- | PIB/polybutylene |
| | | co-vinyl alcohol | |
| B584. | styrene-butadiene | poly(vinylpyrrolidone- | styrene-iosprene |
| | | co-vinyl alcohol | |
| B585. | styrene-butadiene | poly(vinylpyrrolidone- | styrene-butadiene |
| | | co-vinyl alcohol | |
| B586. | styrene-butadiene | alginate | PIB |
| B587. | styrene-butadiene | alginate | silicone polymer |
| B588. | styrene-butadiene | alginate | silicone polymer mixture |
| B589. | styrene-butadiene | alginate | acrylate copolymer |
| B590. | styrene-butadiene | alginate | acrylic ester copolymer |
| B591. | styrene-butadiene | alginate | butyl rubber |
| B592. | styrene-butadiene | alginate | PIB/polybutylene |
| B593. | styrene-butadiene | alginate | styrene-iosprene |
| B594. | styrene-butadiene | alginate | styrene-butadiene |
| B595. | styrene-butadiene | PVA | PIB |
| B596. | styrene-butadiene | PVA | silicone polymer |
| B597. | styrene-butadiene | PVA | silicone polymer mixture |
| B598. | styrene-butadiene | PVA | acrylate copolymer |
| B599. | styrene-butadiene | PVA | acrylic ester copolymer |
| B600. | styrene-butadiene | PVA | butyl rubber |
| B601. | styrene-butadiene | PVA | PIB/polybutylene |
| B602. | styrene-butadiene | PVA | styrene-iosprene |
| B603. | styrene-butadiene | PVA | styrene-butadiene |
| B604. | styrene-butadiene | PVP | PIB |
| B605. | styrene-butadiene | PVP | silicone polymer |
| B606. | styrene-butadiene | PVP | silicone polymer mixture |
| B607. | styrene-butadiene | PVP | acrylate copolymer |
| B608. | styrene-butadiene | PVP | acrylic ester copolymer |
| B609. | styrene-butadiene | PVP | butyl rubber |
| B610. | styrene-butadiene | PVP | PIBPIB/polybutylene |
| B611. | styrene-butadiene | PVP | styrene-iosprene |
| B612. | styrene-butadiene | PVP | styrene-butadiene |
| B613. | styrene-butadiene | water-soluble | PIB |
| | | starch derivative | |
| B614. | styrene-butadiene | water-soluble | silicone polymer |
| | | starch derivative | |
| B615. | styrene-butadiene | water-soluble | silicone polymer mixture |
| | | starch derivative | |
| B616. | styrene-butadiene | water-soluble | acrylate copolymer |
| | | starch derivative | |
| B617. | styrene-butadiene | water-soluble | acrylic ester copolymer |
| | | starch derivative | |
| B618. | styrene-butadiene | water-soluble | butyl rubber |
| | | starch derivative | |
| B619. | styrene-butadiene | water-soluble | PIB/polybutylene |
| | | starch derivative | |
| B620. | styrene-butadiene | water-soluble | styrene-iosprene |
| | | starch derivative | |
| B621. | styrene-butadiene | water-soluble | styrene-butadiene |
| | | starch derivative | |
| B622. | styrene-butadiene | pullulan | PIB |
| B623. | styrene-butadiene | pullulan | silicone polymer |
| B624. | styrene-butadiene | pullulan | silicone polymer mixture |
| B625. | styrene-butadiene | pullulan | acrylate copolymer |
| B626. | styrene-butadiene | pullulan | acrylic ester copolymer |
| B627. | styrene-butadiene | pullulan | butyl rubber |
| B628. | styrene-butadiene | pullulan | PIB/PIB/polybutylene |
| B629. | styrene-butadiene | pullulan | styrene-iosprene |
| B630. | styrene-butadiene | pullulan | styrene-butadiene |
| B631. | styrene-butadiene | water-soluble | PIB |
| | | cellulose derivative | |
| B632. | styrene-butadiene | water-soluble | silicone polymer |
| | | cellulose derivative | |
| B633. | styrene-butadiene | water-soluble | silicone polymer mixture |
| | | cellulose derivative | |
| B634. | styrene-butadiene | water-soluble | acrylate copolymer |
| | | cellulose derivative | |
| B635. | styrene-butadiene | water-soluble | acrylic ester copolymer |
| | | cellulose derivative | |
| B636. | styrene-butadiene | water-soluble | butyl rubber |
| | | cellulose derivative | |
| B637. | styrene-butadiene | water-soluble | PIB/PIB/polybutylene |
| | | cellulose derivative | |
| B638. | styrene-butadiene | water-soluble | styrene-iosprene |
| | | cellulose derivative | |
| B639. | styrene-butadiene | water-soluble | styrene-butadiene |
| | | cellulose derivative | |
| B640. | styrene-butadiene | polyvinyl acetate, | PIB |
| | | p.h. | |
| B641. | styrene-butadiene | polyvinyl acetate, | silicone polymer |
| | | p.h. | |
| B642. | styrene-butadiene | polyvinyl acetate, | silicone polymer mixture |
| | | p.h. | |
| B643. | styrene-butadiene | polyvinyl acetate, | acrylate copolymer |
| | | p.h. | |
| B644. | styrene-butadiene | polyvinyl acetate, | acrylic ester copolymer |
| | | p.h. | |
| B645. | styrene-butadiene | polyvinyl acetate, | butyl rubber |
| | | p.h. | |
| B646. | styrene-butadiene | polyvinyl acetate, | PIB/PIB/polybutylene |
| | | p.h. | |
| B647. | styrene-butadiene | polyvinyl acetate, | styrene-iosprene |
| | | p.h. | |
| B648. | styrene-butadiene | polyvinyl acetate, | styrene-butadiene |
| | | p.h. | |

Most particularly preferred are TTS with one of the combinations of base polymers in the individual layers listed below in table 4:

TABLE 4

| | inner layer | central layer | outer layer |
|---|---|---|---|
| C1. | polyisobutylene, in | PVP, in particular with | polyisobutylene, in |
| | particular with M_{W} = | M_{W} =1,000,000 to | particular with Mw = |
| | about 250,000 g/mol | 1,500,000 g/mol | about 250,000 g/mol |
| C2. | silicone polymer, in | PVP, in particular with | silicone polymer, in |
| | particular a poly- | M_{W} =1,000,000 to | particular a poly- |
| | condensed PDMS/ | 1,500,000 g/mol | condensed |
| | resin network with a | | PDMS/resin network |
| | resin-PDMS weight | | with a resin-PDMS |
| | ratio of 65:35 to | | weight ratio of 65:35 |
| | 55:45 | | to 55:45 |
| C3. | Mixture of a PIB with | PVA, in particular with | Mixture of a PIB with |
| | higher molecular | M_{W} = about 31,000 | higher molecular |
| | weight, in particular | g/mol, P_{W} = about | weight, in particular |
| | M_{W} = about 250,000 | 630, hydrolysis | M_{W} =about 250,000 |
| | g/mol, and a PIB with | degree = about 87 ± 1 | g/mol, and a PIB with |
| | lower molecular | mol % | lower molecular |
| | weight, in particular | | weight, in particular |
| | M_{W} = about 36,000 | | M_{W} = about 36,000 |
| | g/mol | | g/mol |
| C4. | Mixture of a PIB with | PVP, in particular with | Mixture of a PIB with |
| | higher molecular | M_{W} = 1,000,000 to | higher molecular |
| | weight, in particular | 1,500,000 g/mol | weight, in particular |
| | M_{W} = about 250,000 | | M_{W} = about 250,000 |
| | g/mol, and a PIB with | | g/mol, and a PIB with |
| | lower molecular | | lower molecular |
| | weight, in particular | | weight, in particular |
| | M_{W} = about 36,000 | | M_{W} = about 36,000 |
| | g/mol | | g/mol |

The TTS according to the invention preferably consist of a three-layer structure according to one of the examples B1 to B648 and C1 to C4.

The TTS according to the invention preferably comprise one double layer with the combinations of adhesive layer and separation layer described in table 5. In table 5, “PIB”, “silicone polymer”, “silicone polymer mixture”, “acrylate copolymer”, “acrylic ester copolymer”, “butyl rubber”, “polybutylene”, “styrene-iosprene”, “styrene-iosprene”, and “styrene-butadiene” have the same meaning as in table 3.

TABLE 5

| | Adhesive layer | Separation layer |
|---|---|---|
| D1. | PIB | poly(vinylpyrrolidone-co-vinyl |
| | | alcohol |
| D2. | PIB | alginate |
| D3. | PIB | PVA |
| D4. | PIB | PVP |
| D5. | PIB | water-soluble starch |
| | | derivative |
| D6. | PIB | pullulan |
| D7. | PIB | water-soluble cellulose |
| | | derivative |
| D8. | PIB | polyvinyl acetate, p.h. |
| D9. | silicone polymer | poly(vinylpyrrolidone-co-vinyl |
| | | alcohol |
| D10. | silicone polymer | alginate |
| D11. | silicone polymer | PVA |
| D12. | silicone polymer | PVP |
| D13. | silicone polymer | water-soluble starch |
| | | derivative |
| D14. | silicone polymer | pullulan |
| D15. | silicone polymer | water-soluble cellulose |
| | | derivative |
| D16. | silicone polymer | polyvinyl acetate, p.h. |
| D17. | silicone polymer mixture | poly(vinylpyrrolidone-co-vinyl |
| | | alcohol |
| D18. | silicone polymer mixture | alginate |
| D19. | silicone polymer mixture | PVA |
| D20. | silicone polymer mixture | PVP |
| D21. | silicone polymer mixture | water-soluble starch |
| | | derivative |
| D22. | silicone polymer mixture | pullulan |
| D23. | silicone polymer mixture | water-soluble cellulose |
| | | derivative |
| D24. | silicone polymer mixture | polyvinyl acetate, p.h. |
| D25. | acrylate copolymer | poly(vinylpyrrolidone-co-vinyl |
| | | alcohol |
| D26. | acrylate copolymer | alginate |
| D27. | acrylate copolymer | PVA |
| D28. | acrylate copolymer | PVP |
| D29. | acrylate copolymer | water-soluble starch |
| | | derivative |
| D30. | acrylate copolymer | pullulan |
| D31. | acrylate copolymer | water-soluble cellulose |
| | | derivative |
| D32. | acrylate copolymer | polyvinyl acetate, p.h. |
| D33. | acrylic ester copolymer | poly(vinylpyrrolidone-co-vinyl |
| | | alcohol |
| D34. | acrylic ester copolymer | alginate |
| D35. | acrylic ester copolymer | PVA |
| D36. | acrylic ester copolymer | PVP |
| D37. | acrylic ester copolymer | water-soluble starch |
| | | derivative |
| D38. | acrylic ester copolymer | pullulan |
| D39. | acrylic ester copolymer | water-soluble cellulose |
| | | derivative |
| D40. | acrylic ester copolymer | polyvinyl acetate, p.h. |
| D41. | butyl rubber | poly vinylpyrrolidone-co-vinyl |
| | | alcohol |
| D42. | butyl rubber | alginate |
| D43. | butyl rubber | PVA |
| D44. | butyl rubber | PVP |
| D45. | butyl rubber | water-soluble starch |
| | | derivative |
| D46. | butylrubber | pullulan |
| D47. | butyl rubber | water-soluble cellulose |
| | | derivative |
| D48. | butyl rubber | polyvinyl acetate, p.h. |
| D49. | PIB/polybutylene | poly(vinylpyrrolidone-co-vinyl |
| | | alcohol |
| D50. | PIB/polybutylene | alginate |
| D51. | PIB/polybutylene | PVA |
| D52. | PIB/polybutylene | PVP |
| D53, | PIB/polybutylene | water-soluble starch |
| | | derivative |
| D54. | PIB/polybutylene | pullulan |
| D55, | PIB/polybutylene | water-soluble cellulose |
| | | derivative |
| D56. | PIB/polybutylene | polyvinyl acetate, p.h. |
| D57. | styrene-iosprene | poly(vinylpyrrolidone-co-vinyl |
| | | alcohol |
| D58. | styrene-iosprene | alginate |
| D59. | styrene-iosprene | PVA |
| D60. | styrene-iosprene | PVP |
| D61. | styrene-iosprene | water-soluble starch |
| | | derivative |
| D62, | styrene-iosprene | pullulan |
| D63. | styrene-iosprene | water-soluble cellulose |
| | | derivative |
| D64. | styrene-iosprene | polyvinyl acetate, p.h. |
| D65. | styrene-butadiene | poly(vinylpyrrolidone-co-vinyl |
| | | alcohol |
| D66. | styrene-butadiene | alginate |
| D67. | styrene-butadiene | PVA |
| D68. | styrene-butadiene | PVP |
| D69. | styrene-butadiene | water-soluble starch |
| | | derivative |
| D70. | styrene-butadiene | pullulan |
| D71. | styrene-butadiene | water-soluble cellulose |
| | | derivative |
| D72. | styrene-butadiene | polyvinyl acetate, p.h. |

Particular preferred are TTS having the following double layers, listed in table 6:

TABLE 6

| | Adhesive layer | Separation layer |
|---|---|---|
| E1. | polyisobutylene, in particular | PVP, in particular with M_{W} = |
| | with M_{W} = about 250,000 | 1,000,000 to 1,500,000 g/mol |
| | g/mol | |
| E2. | silicone polymer, in particular | PVP, in particular with M_{W} = |
| | a poly-condensed | 1,000,000 to 1,500,000 g/mol |
| | PDMS/resin network with a | |
| | resin-PDMS weight ratio of | |
| | 65:35 to 55:45 | |
| E3. | Mixture of a PIB with higher | PVA, in particular with M_{W} = |
| | molecular weight, in | about 31,000 g/mol, M_{W} = |
| | particular M_{W} = about | about 630, hydrolysis degree = |
| | 250,000 g/mol, and a PIB | about 87 ± 1 mol % |
| | with lower molecular weight, | |
| | in particular M_{W} = about | |
| | 36,000 g/mol | |
| E4. | Mixture of a PIB with higher | PVP, in particular with M_{W} = |
| | molecular weight, in | 1,000,000 to 1,500,000 g/mol |
| | particular M_{W} = about | |
| | 250,000 g/mol, and a PIB | |
| | with lower molecular weight, | |
| | in particular M_{W} = about | |
| | 36,000 g/mol | |

In preferred embodiments, all adhesive layers or the inner and outer layers of a TTS contain identical base polymers. In these cases, the adhesive layers or the inner or outer layers are also preferably identical in their other properties and in their structure.

In a further preferred embodiment, all separation layers of a TTS contain identical base polymers. In these cases, the separation layers are also preferably identical in their other properties and in their structure.

In a particular embodiment, all double layers of the TTS are identical in composition and structure.

The at least one active ingredient of the TTS is preferably distributed homogenously in at least one layer of the TTS. Preferably, the remaining layers control the release of the at least one active ingredient from the TTS. Preferably, all layers of the TTS are permeable for the at least one active ingredient.

In a preferred embodiment, the at least one active ingredient in the central layer is distributed homogenously, and the inner layer is permeable for this at least one active ingredient, and its outer layer is impermeable for this at least one active ingredient. In a particularly preferred embodiment, the outer layer, however, is permeable for the at least one active ingredient.

The TTS contains a total of 5 to 40% (w/w), preferably 10 to 35% (w/w) and particularly preferred 18 to 27% (w/w) active ingredient based on the total weight of the patch without protective foil and backing. It can contain 18% (w/w) or 27% (w/w) active ingredient, for example. In doing so, the active ingredient-containing layer preferably contains at least 45% (w/w), preferably at least 60% and particularly preferred about 69% (w/w) active ingredient.

In an alternative embodiment, the TTS contains less than 9% (w/w), preferably less than 7.5% (w/w) and particularly preferred less than 5% (w/w) active ingredient (in particular rotigotine base) based on the total dry weight of the TTS without protective foil and backing.

Preferably, the inner layer only becomes permeable for the at least one active ingredient during the treatment. Preferably, the inner layer controls the release of the at least one active ingredient from the TTS. Preferably, the inner layer prevents back-diffusion of the at least one active ingredient into the TTS.

Both polar (hydrophilic) and non-polar (lipophilic) active ingredients can be used as active ingredients according to the invention. The at least one active ingredient as free base, salt, or free acid is preferably selected from the group consisting of estradiol, norethisterone acetate, nicotine, fentanyl, sufentanil, tulobuterol, rivastigmine, rotigotine, rasagiline, ethinyl estradiol/norelgestromin, buprenorphine and nitroglycerin, in particular rotigotine or nicotine. In a preferred embodiment the active ingredient is rotigotine.

In case of the use of polyvinylpyrrolidone as base polymer for the central layer or the separation layers, active ingredients are preferably contained, the solubility of which in polyvinylpyrrolidone is at least twice as high compared to the solubility in a 1:1 (wt.) mixture of polyvinylpyrrolidone and water.

Emollient lipophilic active ingredients or mixtures thereof soluble in ethanol at least 0.1 wt.-% at 20° C. can also be contained.

The addition of the active ingredients can basically be implemented into all, only individual ones, or also only into a single one of the layers. In doing so, methods of joint dissolution of active ingredient and excipient with subsequent drying can also be selected just as those methods, wherein the active ingredient itself is used as solvent of the polymer. In the individual case, it can be advantageous (in particular in case of volatile active ingredients) to build up the active ingredient-containing layer two-layered with identical base polymer. In doing so, base material is first dissolved in the active ingredient and applied onto a layer of pure base polymer. The subsequent quick equilibration of the active ingredient creates a homogeneous active ingredient-containing layer.

Optionally, at least one of the layers contains at least one additive. The following additives are possible: Antioxidants, stabilizers, tackifiers, preservatives or penetration enhancers. Whether adding such components to the essential components of the invention, as defined in the claims, is useful in the individual case, can be ascertained by means of routine experiments. These embodiments are therefore explicitly included in the subject matter of the invention.

The permeation properties are advantageously improved by permeation enhancers, which can be selected from the group of fatty alcohols, fatty acids, esters of fatty acids, amides of fatty acids, glycerol or its esters of fatty acids, N-methylpyrrolidone, terpenes such as limonene, α-pinene, α-terpineol, carvone, carveol, limonene oxide, pinene oxide, 1,8-eucalyptol. In one embodiment, the inner layer contains at least one additive, preferably a permeation enhancer.

Within the scope of manufacturing the TTS according to the invention, all layers can be created by means of classical techniques of dissolving, mixing, coating, and temperature-protected drying or simply by means of heat embossing.

To do so, individual layers on provided dehesively equipped support foils generally made of polyethylene terephthalate (PET) can be applied pursuant to methods long known to the person skilled in the art by means of solvent-containing coating methods using blade, slit die, spray, or roller application in uniform layer thicknesses with an application weight after drying preferably not exceeding 70 g/m². In case of double-sided staggered silicone polymerization, it is possible to directly wind with the substrate.

Subsequently, the active ingredient is accepted into a non-adhesive polymer excipient, and a homogeneous inner phase is created from the water-absorbing or water-welling polymer by means of coating on provided dehesively equipped support foils by means of a solvent-containing coating method using blade, slit die, spray, or roller application in uniform layer thicknesses having the above values for the application weight after drying. Furthermore, the application of the hot-melt method is possible, as well.

The manufacturing of these thin layers is possible for the person skilled in the art today with the customary coating, drying, and extrusion methods. In doing so, the addition of the active ingredient can (sic: missing verb in source text, probably: be effected) into one or more layers—central, inner and/or outer layer or separation and/or adhesive layer/s—both solvent-containing after interim drying processes and solvent-free if the active ingredient is liquid at processing temperature or another solvent has been added, which remains in the formulation. The addition to the central layer is the preferred way to faster achieve the equilibrium of the concentration of the active ingredient.

In any case, based on the low diffusion paths, a distribution of the active ingredient into the system components is easily possible, within hours to days after manufacturing if desired.

The layers can be manufactured in any desired sequence and laminated onto one other according to processes known to the person skilled in the art. Without remaining with the system at application, an essentially active ingredient-impermeable backing can be provided, which protects the TTS from adhering to textiles. Furthermore, a re-detachable protective layer can also be provided, which is removed prior to application of the TTS to the skin.

In a preferred embodiment, the manufacturing of the multi-layered TTS according to the invention is implemented with the methods described in DE 101 47 036 A1 and DE 10 2008 038 595 A1. By means of the methods described therein, substrates coated with a protective foil can be laminated particularly advantageously, which achieves a particularly homogeneous application of the adhesive.

In alternative embodiments, however, the following application and lamination systems can also be employed for the manufacturing of the TTS according to the invention:

Knife System; Double Side System; Commabar System; Case Knife System; Engraved Roller System; 2 Roller System; 3 Roller System; Micro Roller System; 5 Roller System;

Reverse Roll System; Rotary Screen System; Dipping System; Slot Die System; Curtain Coating System; Hotmelt Slot Die System; Powder Scattering System.

In a preferred embodiment, the TTS according to the invention is manufactured by means of the so called “slot die” system with a “Smartcoater” (Fa. Coatema Coating Machinery GmbH, Dormagen, Germany), which is based on die technology. In doing so, the die presents a closed application system, which consists of a die chamber, into which the raw lamination material is to be pumped. The geometry of the die, which is determined specifically for any raw lamination material with respect to its process flow diagram, guaranties a homogeneous discharge of the raw lamination material from the discharge slot. A (micro) pump feeds the lamination medium to the pump with high accuracy of dosage. The lamination quantity can be precisely defined by means of the pump speed. Aside from this, the application speed is defined by the discharge slot as well as the speed of the products. Therefore, very thin layers of less than 5 μm are possible depending on the viscosity of the raw material.

### EXAMPLES

The invention is explained in more detail below by means of individual embodiment examples.

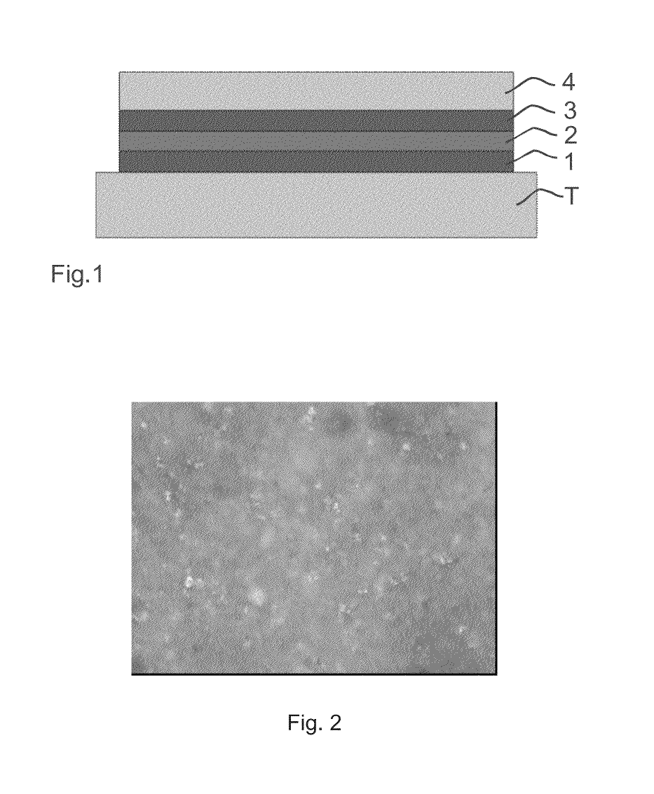

FIG. 1 shows a three-layer transdermal therapy system

T—re-detachable protective foil, to be removed prior to use
1—skin-sided adhesive layer
2—layer with water-soluble polymer
3—adhesive layer between layer 2 and 4
4—active ingredient-impermeable and occlusive backing

FIG. 2 shows a microscopic image of the final TTS from example 3 (polyisobutylene/polyvinylpyrrolidone/polyisobutylene)

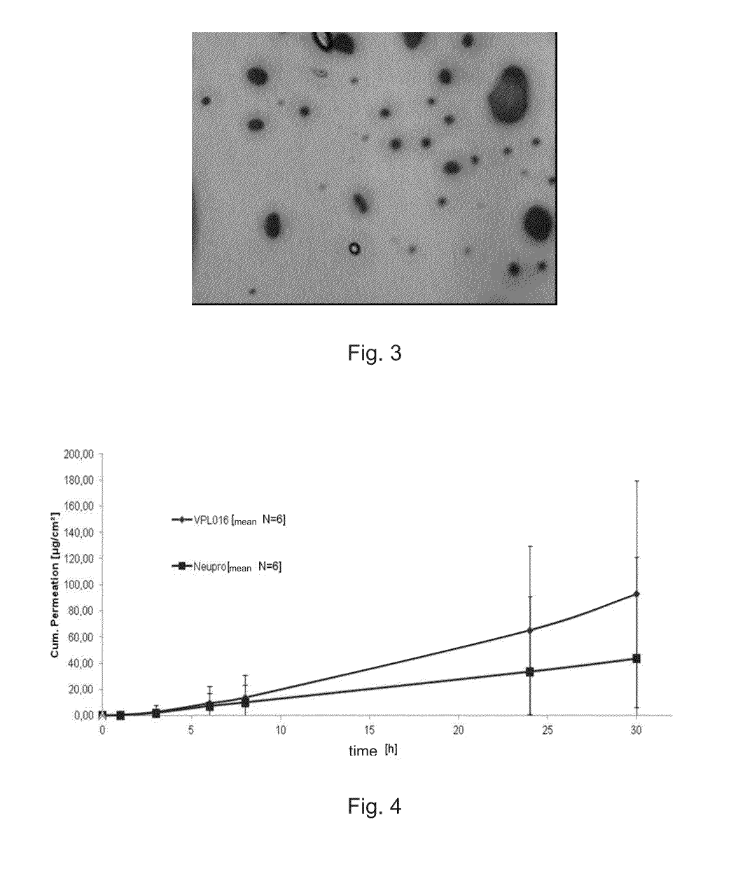

FIG. 3 shows a microscopic image of the final TTS from example 4 (polyisobutylene/polyvinyl alcohol/polyisobutylene)

FIG. 4 shows the pharmacokinetics of the TTS from example 4 (polyisobutylene/polyvinyl alcohol/polyisobutylene)

FIG. 5 shows the water absorption of a polyvinylpyrrolidone polymer (Kollidon) depending on humidity.

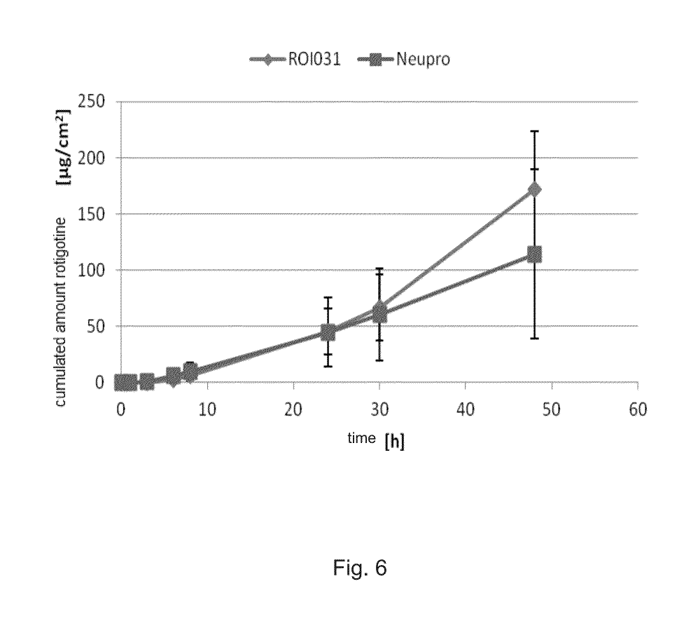

FIG. 6 shows the pharmacokinetics of the TTS from example 6 (silicone adhesive/polyvinylpyrrolidone/silicone adhesive)

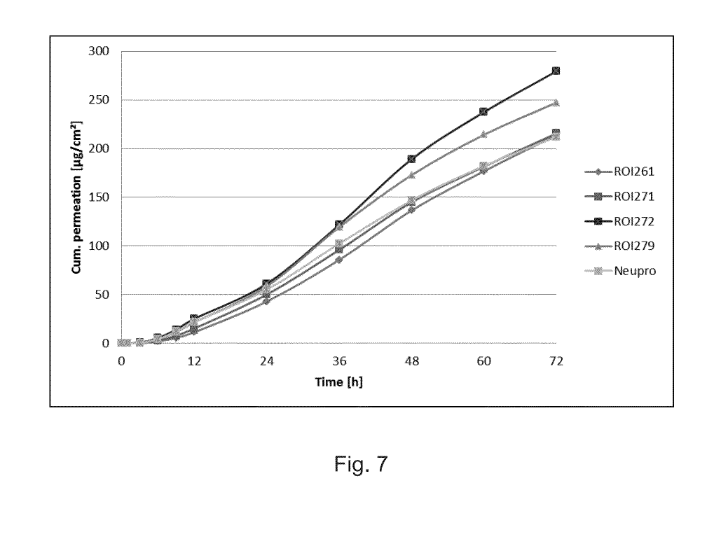

FIG. 7 shows a diagram of the skin permeation over time. The curves represent patches pursuant to examples 12 a) “ROI261”, c) “ROI 271”, d) “ROI 272”, f) “ROI 279”. The curve of the market product “Neupro” is indicated for comparison.

### EXAMPLE 1

### Manufacturing of a System According to the Invention of Polyisobutylene and Polyvinylpyrrolidone with Rotigotine

The PIB used herein is, for example, Oppanol 100 from the manufacturer BASF (Germany) with a weight-average molecular weight of 250,000 g/mol corresponding to a viscosity-average molecular weight of 1.1·10⁶ g/mol.

a) 20 g polyisobutylene is completely diluted in 60 g n-heptane and laminated with a gap width of about 100 μm onto silicone-polymerized PET foil 50 μm. After drying for more than 7 min. at 60 (sic: missing: °) and subsequently 10 min. at 80° C., a homogeneous self-adhesive layer of 25 g/m² is obtained.
b) 20 g polyvinylpyrrolidone (e.g., Kollidon 90 F) and 30 g rotigotine are completely diluted in 120 g ethanol and laminated with a gap width of about 120 μm onto silicone-polymerized PET foil 50 μm. After drying for more than 10 min. at 70° C., a homogeneous non-adhesive layer of 22 g/m² is obtained on the substrate.
c) 20 g polyisobutylene is completely diluted in 60 g n-heptane and laminated with a gap width of about 100 μm onto silicone-polymerized PET foil 75 μm. After drying for more than 7 min. at 60 (sic: missing: °) and subsequently 10 min. at 80° C., a homogeneous self-adhesive layer of 25 g/m² is obtained.
d) A PET foil with a thickness of 20 μm is laminated onto the interim product from a) with the adhesive side, and the silicone-polymerized PET foil 50 μm is removed and discarded.

The formed laminate is laminated onto the interim product from b) with the adhesive side, and the silicone-polymerized PET foil 50 μm is removed and discarded.

The non-adhesive side of the formed bi-laminate originating from interim product b) is laminated onto interim product c) whereby the tri-laminate is formed yielding the silicone-polymerized PET foil 75 μm.

After about 1 hour of equilibration, punched products of 30 cm² are punched. They can be directly adhered to the skin for therapeutic purposes after the silicone-polymerized PET foil 75 μm has been removed and discarded.

### EXAMPLE 2

### Manufacturing of a System According to the Invention of Polydimethylsiloxane and Polyvinylpyrrolidone with Rotigotine

a) 30 g polydimethylsiloxane is completely diluted in 40 g n-heptane and laminated with a gap width of about 100 μm onto fluoropolymer-coated PET foil 50 μm. After drying for 2 min. at 60° and subsequently 2 min. at 80° C., a homogeneous self-adhesive layer of 28 g/m² is obtained.
b) 20 g polyvinylpyrrolidone (e.g., Kollidon 90) and 30 g rotigotine are completely diluted in 120 g ethanol and laminated with a gap width of about 120 μm onto silicone-polymerized PET foil 50 μm. After drying for 10 min. at 70° C., a homogeneous, non-adhesive layer, calculated free of active ingredient, of 12 g/m² is obtained on the substrate.
c) 30 g polydimethylsiloxane is completely diluted in 40 g n-heptane and laminated with a gap width of about 100 μm onto fluoropolymer-coated PET foil 100 μm. After drying for 2 min. at 60 (sic: missing: °) and subsequently 2 min. at 80° C., a homogeneous self-adhesive layer of 28 g/m² is obtained.
d) A PET foil with a thickness of 20 μm is laminated to the interim product from a) with the adhesive-side, and the fluoropolymer-coated PET foil 50 μm is removed and discarded.

The formed laminate is laminated onto the interim product from b) with the adhesive side, and the silicone-polymerized PET foil 50 μm is removed and discarded.

The non-adhesive side of the laminate originating from interim product b) is laminated onto interim product c) with the adhesive side, the silicone-polymerized PET foil 100 μm of the now formed tri-laminate now serves as release liner.

After about 1 hour of equilibration, punched products of 30 cm² are punched. They can be directly adhered to the skin for therapeutic purposes after the fluoropolymer-coated PET foil 100 μm has been removed and discarded.

### Reference Examples for Example 3 and 4

### Manufacturing of Polyisobutylene (PIB) Layers

### Reference Example 1

### PIB Spread on Siliconized Protective Foil

Polyisobutylene (PIB; Durotak 87-6908) is spread onto the siliconized side of a Scotchpak 1022 foil with a desired basis weight of 30 g/m².

Basis weight: 32.412 g/m²
Minimal value: 30.16 mg
Maximum value: 35.16 mg
s (abs.) matrix: 1.79 mg
s (rel.) matrix: 5.54%

The obtained PIB layer can be laminated with the siliconized side of a Scotchpak 1022 foil for interim storage.

### Reference Example 2

### PIB Spread on Backing

PIB (Durotak 87-6908) is spread onto the siliconized side of a Scotchpak 9738 foil with a desired basis weight of 30 g/m².

Basis weight: 38.142 g/m²
Minimal value: 29.32 mg
Maximum value: 61.48 mg
s (abs.) matrix: 11.56 mg
s (rel.) matrix: 30.30%

The obtained PIB layer can be laminated with the siliconized side of a Scotchpak 1022 foil for interim storage.

### EXAMPLE 3

### Manufacturing of a TTS with a Rotigotine-Containing Polyvinyl Alcohol (PVA) Layer

### Manufacturing of Rotigotine-Containing PVA Composition

The PVA used herein is, for example, Mowiol 4-88 from the manufacturer Clariant (Germany). This is a partially hydrolyzed PVA with a weight-average molecular weight Mw of 31,000 g/mol, an average degree of polymerization Pw of 630, and a degree of hydrolysis of 87±1 mol %.

6 g PVA is dissolved in 22 g aqua purificata under stirring and heating to 90° C. over night. 22 g absolute ethanol is carefully and slowly added by dripping and stirred until a homogeneous composition has formed. 13.5 g rotigotine base is scattered and incorporated under heating at 70° C. It is stirred until a homogeneous composition has formed. The composition is allowed to cool, and evaporated ethanol (98%) is added.

Spreading the Rotigotine-Containing PVA Composition onto Protective Foil

The composition thus obtained is spread onto the siliconized side of a Scotchpak 1022 foil with a desired basis weight of 30 g/m² and is dried for 20 min at 70° C. The formed rotigotine-containing PVA layer can be laminated with the siliconized side of a Scotchpark 1022 foil for interim storage.

The laminate of rotigotine-containing PVA-layer and Scotchpak 1022 foil is first laminated together with the laminate of PIB-layer and backing (see reference example 2), and the thus formed laminate, in turn, is laminated with the laminate of PIB-layer and protective foil (reference example 1) to result in the final TTS, which is punched out and microscoped (see FIG. 2).

The manufactured TTS appears white and contains a total of 9% (w/w) rotigotine based on the base and 24% PVA. The central rotigotine-containing PVA-layer contains 27% (w/w) rotigotine based on the base and 73% PVA.

### EXAMPLE 4

### Manufacturing of a TTS with a Rotigotine-Containing Polyvinylepyrrolidone (PVP) Layer

### Manufacturing of a Rotiqotine-Containing PVP Composition

The PVP used herein is, for example, Kollidon 90 F from the manufacturer BASF (Germany) with a weight-average molecular weight Mw of 1,000,000 to 1,500,000 g/mol.

100 g of a 25% solution of annealed Kollidon in absolute ethanol is manufactured under stirring over night. Another 12.5 g absolute ethanol is added to 24 g of this Kollidon solution. 13.5 g rotigotine base is scattered and stirred in a water bath for 90 min. at 60° C. until a homogeneous clear solution has formed. It is further cold-stirred for 30 min. and evaporated absolute ethanol is added.

Spreading of the Rotiqotine-Containing PVP Composition onto Protective Foil

The thus formed composition is spread onto the non-siliconized side of a Silphan foil (Silikonnatura) with a basis weight of 45.103 g/m² and dried for 20 min. at 70° C. The formed rotigotine-containing PVP layer can be laminated with the siliconized side of a Scotchpak 1022 foil for interim storage.

The laminate of rotigotine-containing PVP layer and Silphan foil is first laminated together with the laminate of PIB layer and backing (see reference example 2), and the thus formed laminate, in turn, is laminated with the laminate of PIB layer and protective foil (reference example 1) to result in the final TTS, which is punched out and microscoped (see FIG. 3).

The manufactured TTS appears colorless and contains a total of 9% (w/w) rotigotine based on the base and 24% (w/w) PVP. The average rotigotine-containing PVP layer contains 27% (w/w) rotigotine based on the base and 73% PVP.

### EXAMPLE 5

### Substance Delivery of the TTS from Example 4

The cumulative permeation depending on the treatment time was determined for the TTS according to the invention, which were manufactured according to example 4, and for micro-reservoir-containing TTS according to DE 603 04 477 T2, respectively. 6 TTS were examined, respectively. In doing so, it was surprisingly found that the rotigotine flux in case of the TTS (VPL016) according to the invention compared to those according to DE 603 04 477 T2 was elevated.

### EXAMPLE 6

### Manufacturing of a TTS with a Rotigotine-Containing Polyvinylpyrrolidone (PVP) Layer and Two Silicone Layers

### Manufacturing of a Rotiqotine-Containing PVP Composition

The PVP used herein is Kollidon 90 F from the 10-30-20 manufacturer BASF (Germany).

5 g Kollidon 90F was added to 15 g absolute ethanol and dissolved therein under stirring over night. 1.329 g rotigotine base was added to this solution and stirring was continued until an optically homogeneous composition had developed.

### Spreading and Drying of the Rotigotine-Containing PVP Layer

The thus obtained composition was spread onto the siliconized side of a release liner (Silphan 75 foil, Fa. Silikonnatura) with a gap width of 170 μm and dried for 20 min. at 50° C.

### Manufacturing of a Silicone Layer

In doing so, the amine-compatible medium tack silicone adhesive Bio Pas 7-4202 (Fa. Dow Corning) was applied to a Scotchpak 1022 foil by means of a Smart Coater (Fa. Coatema, Federal Republic of Germany) as release liner/backing foil and subsequently dried in three steps at 80° C., 90° C., and 90° C. The dried silicone layer had a basis weight of 19.6 g/m².

### EXAMPLE 7

### Manufacturing of a TTS with a Rotigotine-Containing Polyvinylpyrrolidone (PVP) Layer and Two Silicone Layers

### Manufacturing of a Rotiqotine-Containing PVP Composition

The PVP used herein is Kollidon 90 F from the manufacturer BASF (Germany).

5 g Kollidon 90F was added to 15 g absolute ethanol and dissolved therein under stirring over night. 1.329 g rotigotine base was added to this solution and stirring was continued until an optically homogeneous composition had developed.

### Spreading and Drying of the Rotigotine-Containing PVP Layer

The thus obtained composition was spread onto the siliconized side of a release liner (Silphan 75 foil, Fa. Silikonnatura) with a gap width of 170 μm and dried for 20 min. at 50° C.

### Manufacturing of a Silicone Oil-Containing Silicone Layer

In doing so, the medium tack silicone adhesive Bio Pas 7-4502 (Fa. Dow Corning) was mixed with the silicone oil under the following conditions. The oil-containing silicone adhesive was applied to a Scotchpak 1022 foil by means of a Smart Coater (Fa. Coatema, Federal Republic of Germany) as release liner/backing foil and subsequently dried in three steps at 80° C., 90° C., and 90° C. The dried silicone had a basis weight of 19.6 g/m².

The laminate of rotigotine-containing PVP layer and Silphan foil is first laminated together with the laminate of silicone layer and backing, and the thus formed laminate, in turn, is laminated onto the laminate of silicone layer and protective foil, to result in the final TTS with a basis weight of 73.5 g/m².

The manufactured TTS appears colorless and contains a total of 9.2% (w/w) rotigotine base and 24% (w/w) PVP. The central rotigotine-containing PVP layer contains 20% (w/w) rotigotine and 80% (w/w) PVP.

The laminate of rotigotine-containing PVP layer and Silphan foil is first laminated together with the laminate of silicone layer and backing, and the thus formed laminate, in turn, is then laminated with the laminate of silicone layer and protective foil, to result in the final TTS with a total basis weight of 73.5 g/m².

The manufactured TTS appears colorless and contains a total of 9%.2% (w/w) rotigotine base and 24% (w/w) PVP. The central rotigotine-containing PVP layer contains 20% (w/w) rotigotine base and 80% (w/w) PVP.

### EXAMPLE 7

### Substance Delivery of the TTS from Example 6 According to the Invention

The cumulative permeation depending on the treatment time was determined for the TTS according to the invention, which were manufactured according to example 5, and for micro-reservoir-containing TTS according to EP 1524975 B9, respectively. Surprisingly, it was found that the rotigotine flux in case of the TTS (ROIO31) according to the invention was identical for 30 hours compared to those according to EP 1524975 B9 (Neupro[R]), but has an increased permeation in the time period from 30 to 48 h (see FIG. 6). One reason for the unusual increase in performance can be found in the water absorption of the PVP, which impairs the saturation solubility of the rotigotine in the central PVP layer and thus increases thermodynamic activity.

### EXAMPLE 8

### Multi-Layer TTS with a Rotigotine-Containing Substance Layer and Two Adhesive Layers

### a) Multi-Layer TTS “PIB 1”

The multi-layer TTS consists of three layers, wherein the central layer, the substance layer, and the two outer layers are adhesive layers. The substance layer contains rotigotine base, a PVP with a weight-average molecular weight of 1,000,000 g/mol, in particular Kollidon 90F (BASF) and an additive, for example butylhydroxytoluene. The adhesive layers contain a PIB-adhesive of PIB with a number-average Mw of 600,000 g/mol, PIB with a weight-average Mw of about 51,000 g/mol, and polybutene of a polybutene with a number-average Mw of about 370 g/mol, in particular the PIB adhesive “DK”.

### b) Multi-Layer TTS “PIB 2”

The multi-layer TTS corresponds to that of a) with the difference that the adhesive layers contain the PIB adhesive Durotak 6908.

### c) Multi-Layer TTS “Silicone 1”

The multi-layer TTS corresponds to that of a) with the difference that the adhesive layers contain an average-adhesive silicone adhesive, in particular BIO-PSA 7-4502.

### d) Multi-Layer TTS “Silicone 2”

The multi-layer TTS corresponds to that of a) with the difference that the adhesive layers contain a highly adhesive silicone adhesive, in particular BIO-PSA 7-4602.

### d) Multi-Layer TTS “Silicone 3”

The multi-layer TTS corresponds to that of a) with the difference that the adhesive layers contain an average-adhesive and a highly adhesive silicone adhesive, in particular BIO-PSA 7-4502 and BIO-PSA 7-4602.

### e) Multi-Layer TTS “Silicone 4”

The multi-layer TTS corresponds to that of a) with the difference that the adhesive layers contain an average-adhesive silicone adhesive, in particular BIO-PSA 7-4502 and a silicone oil, in particular Dow Corning Q7-9120 type 1000.

### EXAMPLE 9

### Multi-Layer TTS with a Rotigotine-Containing Substance Layer and Two Adhesive Layers

### a) Multi-Layer TTS “R01261”

The multi-layer TTS consists of three layers, wherein the central layer, the substance layer and the two outer layers are adhesive layers. The layers each have a basis weight of 20 g/cm². The substance layer consists of 4.5 mg rotigotine base, 15.38 mg PVP with a weight-average molecular weight of 1,000,000 g/mol, in particular Kollidon 90F (BASF), and 0.12 mg additive, for example butylhydroxytoluene. The adhesive layers each consist of 10 mg of a PIB adhesive, in particular “DK” (23.4 wt.-% Oppanol B 100+33.5 wt.-% Oppanol B12+43 wt.-% Indopol L14 (polybutene))

### b) Multi-Layer TTS “ROI262”

The multi-layer TTS corresponds to that of a) with the difference that the adhesive layers each consist of 10 mg PIB adhesive Durotak 6908. [Which is why, . . . d

### c) Multi-Layer TTS “ROI271”

The multi-layer TTS corresponds to that of a) with the difference that the adhesive layers each consist of 20 mg of an average-adhesive silicone adhesive, in particular BIO-PSA 7-4502.

### d) Multi-Layer TTS “ROI272”

The multi-layer TTS corresponds to that of a) with the difference that the adhesive layers each consist of 20 mg of a highly adhesive silicone adhesive, in particular BIO-PSA 7-4602.

### d) Multi-Layer TTS “ROI273”

The multi-layer TTS corresponds to that of a) with the difference that the adhesive layers each consist of 10 mg of an average-adhesive and 10 mg of a highly adhesive silicone adhesive, in particular BIO-PSA 7-4502 and BIO-PSA 7-4602.

### e) Multi-Layer TTS “ROI279”

The multi-layer TTS corresponds to that of a) with the difference that the adhesive layers each consist of 19.8 mg of an average-adhesive silicone adhesive, in particular BIO-PSA 7-4502, and 0.2 mg of a silicone oil, in particular Dow Corning Q7-9120 type 1000.

1. Transdermal therapy system (TTS), characterized in that it comprises
(i) a central layer, which contains at least one active ingredient,
(ii) an inner layer, which is permeable for the at least one active ingredient, and
(iii) an outer layer
as well as optionally a backing and/or a protective foil, wherein the central layer contains at least one hygroscopic polymer or copolymer.

2. TTS according to claim 1, wherein the hygroscopic polymer or copolymer has a weight proportion of 50% or more, preferably of 60, 70 or 80% or more, particularly preferred of 90% or more, and in particular of 100% based on the weight of the total polymer of the central layer.

3. TTS according to claim 1 or 2, wherein none of the layers has a basis weight of more than 45, preferably of more than 30 g/m².

4. TTS according to claim 1 to 3, wherein the basis weights of the central and the inner layer are in a ratio of 5:1 to 1:5, preferably of 3:1 to 1:3, further preferred of 3:2, and particularly preferred of 1:1.

5. TTS according to one of the previous claims, wherein the basis weights of the inner and the outer layer are in a ratio of 5:1 to 1:5, preferably of 3:1 to 1:2, particularly preferred of 2:1.

6. TTS according to one of the previous claims, wherein the basis weights of the central and the outer layer are in a ratio of 5:1 to 1:5, preferably of 4:1 to 1:2, particularly preferred of 3:1.

7. TTS according to one of the previous claims, wherein the at least one hygroscopic polymer or copolymer has a water absorption of at least 15 wt.-%, preferably at least 25 wt.-%, and particularly preferred at least 35 wt.-% based on its own weight at 75% relative humidity, 25° C. and 1013.25 hPa after saturation.

8. TTS according to one of the previous claims, wherein the at least one polymer or copolymer of the central layer is more than 60% (w/w), preferably more than 70% (w/w), further preferred more than 80% (w/w), and particularly preferred more than 90% (w/w) of at least one hygroscopic polymer or copolymer.

9. TTS according to one of the previous claims, wherein the at least one hygroscopic polymer or copolymer is polyvinylpyrrolidone, polyvinyl alcohol, partially hydrolyzed polyvinyl acetate, poly(vinylpyrrolidone-co-vinyl alcohol) and/or a polysaccharide, preferably a water-soluble starch derivative, a water-soluble cellulose derivate, pullulan or an alginate.

10. TTS according to claim 9, wherein the at least one hygroscopic polymer is polyvinylpyrrolidone or polyvinyl alcohol.

11. TTS according to one of the previous claims, wherein the inner and/or the outer layer contain at least one hydrophobic polymer, preferably a mixture of various polyisobutylenes and/or a silicone polymer or a mixture of various silicone polymers.

12. TTS according to one of the previous claims, wherein the layers (ii) and (iii) contain identical polymers.

13. TTS according to one of the previous claims, wherein the central layer is provided as separation layer, i.e., has a tensile strength of at least 0.1 N/mm², preferably of at least 2 N/mm².

14. TTS according to one of the previous claims, wherein the inner and/or the outer layer is provided as adhesive layer, i.e., has an adhesive force (to steel) of at least 1 N/cm², preferably of at least 10 N/cm².

15. TTS according to one of the previous claims, comprising at least two, preferably at least three double layers and optionally a further adhesive layer, and the double layers respectively consisting of a separation layer and an adhesive layer.

16. TTS according to one of the previous claims, wherein the at least one active ingredient is homogenously distributed.

17. TTS according to one of the previous claims, wherein the at least one active ingredient is selected as free base, free acid or salt from the group consisting of estradiol, norethisterone acetate, nicotine, fentanyl, sufentanil, tulobuterol, rivastigmine, rotigotine, rasagiline, ethinyl estradiol/norelgestromin, buprenorphine and nitroglycerin, in particular from the group consisting of rotigotine and nicotine.
